(19) United States
(12) Patent Application Publication
(10) Pub. No.: US 2026/0248953 A1
(43) Pub. Date: Aug. 27, 2026

(54) METHOD OF TREATING CANCER CELLS WITH A FOLIC ACID AND CARBOPLATIN FUNCTIONALIZED NICKEL FERRITE/MONODISPERSED SPHERICAL SILICA NANOCOMPOSITE

(71) Applicant: Imam Abdulrahman Bin Faisal University, Dammam (SA)

(72) Inventors: B. Rabindran JERMY, Dammam (SA); Vijaya RAVINAYAGAM, Dammam (SA); Munther ALOMARI, Dammam (SA)

(73) Assignee: Imam Abdulrahman Bin Faisal University, Dammam (SA)

(21) Appl. No.: 19/062,160

(22) Filed: Feb. 25, 2025

(51) Int. Cl. A61K 47/69; A61K 47/52; B82Y 5/00

(52) U.S. Cl. CPC A61K 47/6929; A61K 47/52; B82Y 5/00

(57) ABSTRACT

A method of treating cancer cells to achieve apoptosis including contacting the cancer cells with a porous particulate nanocomposite in an amount sufficient to kill the cancer cells. The porous particulate nanocomposite contains a magnetic nickel ferrite (NiFe₂O₄) having an inverse spinel crystal structure; and, monodisperse spherical silica (Sil) particles onto which the magnetic NiFe₂O₄ is dispersed. The nanocomposite is functionalized with cis-diammine (cyclobutane-1,1-dicarboxylate-O,O′)platinum(II) (carboplatin or Carbpt) and folic acid (FA). The cancer cells are cells of colon cancer, colorectal cancer and/or cervical cancer.

### BACKGROUND

### Technical Field

The present disclosure is directed towards a nanocomposite, and more particularly, towards a folic acid (FA) and cis-diammine(cyclobutane-1,1-dicarboxylate-O,O′)platinum(II) (carboplatin or Carbpt) functionalized nickel ferrite (NiFe₂O₄)/monodispersed spherical silica (Sil) porous particulate nanocomposite (NiFe₂O₄/Sil/FA/Carbpt) having utility in the killing of cancer cells.

### Description of Related Art

The ‘background’ description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Colon cancer is one of the most dominant cancers globally, ranking third in diagnosis and causing approximately six hundred thousand deaths annually. Existing treatments, including chemotherapy and surgical interventions, are limited by challenges such as suboptimal drug adsorption, dose-dependent toxicity, drug resistance, and a lack of selectivity between normal and cancerous cells. [See: Yi, X. et al., A step-by-step multiple stimuli-responsive metal-phenolic network prodrug nanoparticles for chemotherapy, Nano Research, 15, pp. 1205-1212 (2022]. These limitations highlight the urgent need for advanced, targeted therapeutic solutions.

Drugs such as 5-fluorouracil (5-FU), capecitabine, oxaliplatin, and irinotecan are widely used for colon cancer treatment. However, these therapies suffer from severe side effects, resistance development, and limited specificity. For instance, 5-fluorouracil requires multiple injections due to its short half-life, leading to frequent exposure of both normal and cancerous cells, which increases toxicity [See: Ugorji, O. L. et al., Engineering 5-flourouracil and leucovorin-loaded vesicular systems for possible colon specific delivery: In vitro evaluation and real time cell assay against HCT-116 colon cell lines, Heliyon, 10 (8), 2024]. Cervical cancer accounted for 570,000 cases with estimated death of about 311,000 women. The standard treatment strategies include chemoradiotherapy and immunotherapy. Until now, the treatment remains unsatisfactory owing to the relapse, reaching metastasis stage within shorter period and reduced survival rate. For such cases of relapse or metastasis, treatment includes combinational chemotherapy drugs including cisplatin (Cispt), paclitaxel and bevacizumab [See: Zhou, P. et al., Nanoparticle-based applications for cervical cancer treatment in drug delivery, gene editing, and therapeutic cancer vaccines, Wiley Interdisciplinary Reviews: Nanomedicine and Nanobiotechnology, 13 (5): 1718 (2021)]. Despite their effectiveness, conventional combinational regimens lack specificity, leading to off-target effects. Additionally, the high systemic toxicity and the emergence of drug resistance further limit their therapeutic potential. Innovative, targeted approaches are urgently required to overcome these limitations [See: Sharma S. et al., Current treatment for cervical cancer: an update. Anti-Cancer Agents in Medicinal Chemistry (Formerly Current Medicinal Chemistry-Anti-Cancer Agents), 20 (15): 1768-1779 (2020)].

The use of nanotechnology offers promising advancements in cancer treatment by enabling precise targeting of cancer cells, thereby minimizing toxicity to healthy tissues. In the case of colon cancer, nanotechnology provides an effective solution for targeted drug delivery to the colon, enhancing chemotherapy drug penetration, optimizing release profiles, improving bioavailability, and significantly increasing therapeutic efficacy at the tumor site. Oral administration of nanoformulations for colon cancer treatment offers advantages, including reduced drug exposure, enhanced safety, and convenience. However, developing an effective drug delivery system (DDS) faces challenges due to complex physiological barriers, poor drug solubility, pH variations, and food interactions within the human body, which can limit the bioavailability and therapeutic efficacy of the drug [See: Wang, N., Current Advances of Nanomaterial-Based Oral Drug Delivery for Colorectal Cancer Treatment, Nanomaterials, 14 (7): 557 (2024)]. The intestinal mucus layer plays a critical role in trapping and eliminating foreign particles, including toxins, pathogens, and drugs, through adhesion and spatial barriers. To overcome this, the drug delivery system (DDS) must be designed to improve drug penetration, bypass the intestinal mucus barrier, and target cancer cells while sparing healthy cells. Colonic DDS may be optimized based on physiological conditions, such as colon pH, microbiota, and time. Eudragit polymer enables pH-triggered drug release, while phloral employs a dual-triggered system based on both pH and microbiota metabolism [See: Laura E. McCoubrey et al., Colonic drug delivery: Formulating the next generation of colon-targeted therapeutics, Journal of Controlled Release 353:1107-1126 (2023)].

To enhance the effectiveness of chemotherapy agents like carboplatin, it is crucial to incorporate advanced DDS that overcome physiological barriers and improve targeted drug release. Carboplatin (Carbpt) is a platinum-based chemotherapy agent used to treat various cancers and which was developed to minimize the toxic effects of Cispt. Carbpt is preferred over Cispt for patients with impaired renal function, hyperhydration, and neuro/ototoxicity. It is widely used in treating small-cell and non-small-cell lung cancers but is less effective for tumors in the ovaries, head, neck, and bladder. Although Carbpt has lower toxicity than Cispt at equimolar concentrations, higher doses are required for treating ovarian, testicular, and lung cancers [See: Bahremand, K. et al., Enhancing cisplatin efficacy with low toxicity in solid breast cancer cells using pH-charge-reversal sericin-based nanocarriers: development, characterization, and in vitro biological assessment, ACS Omega, 9 (12): 14017-14032 (2024)].

Niosomes, non-ionic surfactant-based vesicles in dehydrated form, have been reported to exhibit a better therapeutic efficiency compared to hydrated niosomes for targeted drug delivery in the colon. However, the use of such a delivery system is often limited by the rapid drug clearance, limited bioavailability and the difficulty in controlling the drug release [See: Sharafshadeh O. S., Preparation and physicochemical properties of cisplatin and doxorubicin encapsulated by noisome alginate nanocarrier for cancer therapy, Int. J. Biol. Macromol. 235:123686 (2023)]. Therefore, delivering drugs to the colon site often requires the integration of multiple delivery techniques to improve targeted drug delivery efficacy. Researchers has developed a composite of 5-Fluorouracil-leucovorin loaded proniosomes encapsulated in eudragit polymer and examined against HCT-116 colon cancer cell lines [See: Ugorji O. L. et al., Heliyon, 10 (8). (2024)]. The formulation showed a lower drug release under simulated gastric pH of 1.2, about 11% at pH 6.8, while more than 80% release at pH 7.4. The proniosomes/Eudragit combo also showed a colon specific in vitro cytotoxicity. Liposomes application in the treatment of colon cancer has attracted considerable attention due to their similar membrane features and high biocompatibility [See: Sang, R. et al., Liposome technologies towards colorectal cancer therapeutics, Acta Biomaterialia, 127:24-40 (2021)]. The combination of acidity triggered rational membrane peptide (ATRAM), liposome, Platycodin D2 (saponin) was found target the tumor site in colorectal cancer xenografts and reactivate the apoptosis to cause cancer cell death [See: Cho E. et al., Tumor-targeted liposomes with platycodin D2 promotes apoptosis in colorectal cancer, Materials Today Bio, p. 100745 (2023)]. Plant lectin protein (peanut agglutinin), topoisomerase I inhibitor (Irinotecan hydrochloride) and chemodrug (capecitabine) loaded liposome was reported to enhance the apoptosis in in vivo and inhibit the colorectal cancer cell proliferation [See: Diao, W. et al., PNA-modified liposomes improve the delivery efficacy of CAPIRI for the synergistic treatment of colorectal cancer, Frontiers in Pharmacology, 13:893151 (2022)].

Liposomes and the biodegradable polymer poly (lactic-co-glycolic acid) (PLGA) composites have also been shown to effectively release Cispt for cervical cancer treatment. Cisplatin was functionalized with PLGA using a double emulsion solvent evaporation technique and encapsulated in anti-VEGF antibody-conjugated liposomes. The therapeutic efficiency was evaluated in 3D cell culture and in vivo with tumor-bearing mice, showing significant anti-tumor effects and cytotoxicity to VEGF-expressing cells [See: Dana P. et al., Active targeting liposome-PLGA composite for cisplatin delivery against cervical cancer, Colloids and Surfaces B: Biointerfaces, 196:111270 (2020)].

Existing cancer treatment methods face limitations, including non-selectivity, toxicity, and the development of drug resistance. Nanocomposites present a promising approach by enabling targeted drug delivery and enhanced therapeutic efficacy. However, challenges such as stability, controlled release, and biocompatibility must be resolved. Addressing these limitations will maximize the potential of nanocomposite-based therapies in cancer treatment. Accordingly, one object of the present disclosure is to provide a porous particulate nanocomposite for the treatment of cancer cells, which may circumvent the drawbacks and limitations, such as non-targeted drug delivery, poor bioavailability, rapid drug clearance, dose-related toxicity, and drug resistance, of the materials and methods already known in the art.

### SUMMARY

In an exemplary embodiment, there is provided a method of treating cancer cells to achieve apoptosis, comprising: contacting the cancer cells with a porous particulate nanocomposite in an amount sufficient to kill the cancer cells, wherein the porous particulate nanocomposite comprises: a magnetic nickel ferrite (NiFe₂O₄); and, monodisperse spherical silica particles onto which the magnetic nickel ferrite (NiFe₂O₄) is dispersed. The porous particulate nanocomposite is functionalized with cis-diammine(cyclobutane-1,1-dicarboxylate-O,O′)platinum(II) (carboplatin) and folic acid. Further, the magnetic nickel ferrite (NiFe₂O₄) has an inverse spinel crystal structure. The cancer cells are cells of a cancer selected from the group consisting of colon cancer, colorectal cancer and cervical cancer.

In some embodiments, the NiFe₂O₄ is superparamagnetic.

In some embodiments, the nanocomposite has a surface area of from about 30 square meter per gram (m²/g) to about 50 m²/g, as determined by Brunauer-Emmett-Teller (BET) analysis.

In some embodiments, the nanocomposite has a surface area of from about 30 m²/g to about 40 m²/g, as determined by BET analysis.

In some embodiments, the nanocomposite has a median pore diameter of from about 10 nanometer (nm) to about 20 nm, as determined by Barrett-Joyner-Halenda (BJH) desorption analysis.

In some embodiments, the nanocomposite has a median pore diameter of from about 15 nm to about 20 nm, as determined by BJH desorption analysis.

In some embodiments, the functionalized nanocomposite has a pore volume of from about 0.1 cubic centimeter per gram (cm³/g) to about 0.3 cm³/g, as determined by BJH desorption analysis.

In some embodiments, the functionalized nanocomposite has a pore volume of from about 0.1 cm³/g to about 0.2 cm³/g, as determined by BJH desorption analysis.

In some embodiments, the Carbpt is present in an amount of from about 1 weight percent (wt. %) to about 10 wt. %, based on the weight of the nanocomposite.

In some embodiments, the Carbpt is present in an amount of from about 3 wt. % to about 7 wt. %, based on the weight of the nanocomposite.

In some embodiments, the folic acid (FA) is present in an amount of from about 1 wt. % to about 10 wt. %, based on the weight of the nanocomposite.

In some embodiments, the folic acid (FA) is present in an amount of from about 3 wt. % to about 7 wt. %, based on the weight of the nanocomposite.

In some embodiments, the nanocomposite includes amide bonds, as determined by Fourier transform infrared spectroscopy (FTIR).

In some embodiments, the FTIR spectrum of the functionalized nanocomposite has an Si—OH peak of lower intensity than that of an FTIR spectrum of a porous particulate nanocomposite of magnetic NiFe₂O₄ and monodisperse spherical Sil particles which is functionalized only with folic acid (FA).

In some embodiments, in accordance with the Korsmeyer-Peppas equation:

$${M}_{t} / {M}_{∞} = k . {t}^{n}$$

in which, Mₜ/M_{∞} represents the fraction of Carbpt released from the nanocomposite at time, the functionalized nanocomposite has, in a phosphate buffered solution (PBS) at a pH of about 7.4: a release constant k of from about 5 to about 30 h⁻ⁿ; and, a transport exponent n of from about 0.05 to about 0.30.

In some embodiments, the nanocomposite has in a phosphate buffered solution (PBS) at a pH of about 7.4: a release constant k of from about 10 h⁻ⁿ to about 20 h⁻ⁿ; and, a transport exponent n of from about 0.05 to about 0.15.

In another exemplary embodiment, a method of preparing the functionalized nanocomposite is described. The method comprises: calcining a comminuted solid mixture of a nickel (II) salt, an iron (III) salt and silica (SiO₂) at a temperature of from about 700° C. to about 1000° C. to form a particulate nanocomposite of magnetic NiFe₂O₄ and monodisperse spherical Sil particles; under mixing, adding in a dropwise manner a solution of folic acid (FA) in a phosphate buffered saline solution to the particulate nanocomposite to form a paste; drying the paste at a temperature of from about 20°° C. to about 50° C. to form a FA functionalized particulate nanocomposite; under mixing at a temperature of from about −10° C. to about 20° C., adding a solution of Carbpt in normal saline solution to the FA functionalized particulate nanocomposite to form a dispersion of the particulate nanocomposite functionalized with FA and Carbpt; and, separating the functionalized particulate nanocomposite from the dispersion.

In some embodiments, the nickel (II) salt is selected from the group consisting of nickel sulfate (NiSO₄), nickel nitrate (Ni(NO₃)₂), nickel chloride (NiCl₂) and nickel acetate (Ni(CH₃COO)₂); and, the iron (III) salt is selected from the group consisting of iron sulfate (Fe₂(SO₄)₃), iron nitrate (Fe(NO₃)₃), iron chloride (FeCl₃) and iron acetate (Fe(CH₃COO)₃).

In yet another exemplary embodiment, there is described a method of providing treatment of at least one condition selected from the group includes colon cancer, colorectal cancer and cervical cancer including administering a functionalized porous particulate nanocomposite to a subject in need of treatment. The functionalized porous particulate nanocomposite comprises: a magnetic nickel ferrite (NiFe₂O₄); and, monodisperse spherical silica particles onto which the magnetic nickel ferrite (NiFe₂O₄) is dispersed, wherein: the porous particulate nanocomposite is functionalized with cis-diammine(cyclobutane-1,1-dicarboxylate-O,O′)platinum(II) (carboplatin) and folic acid; and, the magnetic nickel ferrite (NiFe₂O₄) has an inverse spinel crystal structure.

In some embodiments, there is described a pharmaceutical composition comprising the functionalized nanocomposite as described hereinabove and at least one pharmaceutically acceptable excipient.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic flow chart depicting a method of synthesizing a folic acid (FA) and cis-diammine(cyclobutane-1,1-dicarboxylate-O,O′)platinum(II) (carboplatin or Carbpt) functionalized nickel ferrite (NiFe₂O₄)/monodispersed spherical silica (Sil) porous particulate nanocomposite (NiFe₂O₄/Sil/FA/Carbpt), according to certain embodiments.

FIG. 2 shows X-ray diffraction (XRD) patterns of Carbpt, FA, Sil/Carbpt, NiFe₂O₄/Sil/Carbpt and NiFe₂O₄/Sil/FA/Carbpt, according to certain embodiments.

FIG. 3A depicts the nitrogen (N₂) adsorption-desorption isotherm of Sil, NiFe₂O₄, NiFe₂O₄/Sil and NiFe₂O₄/Sil/FA/Carbpt, according to certain embodiments.

FIG. 3B depicts the pore size distribution curve for Sil and Sil/FA/Carbpt, according to certain embodiments.

FIG. 4 illustrates the diffuse reflectance spectroscopic (DRS) images of Carbpt, FA, Sil/Carbpt, NiFe₂O₄/Sil/Carbpt and NiFe₂O₄/Sil/FA/Carbpt, according to certain embodiments.

FIG. 5A shows a scanning electron microscopy (SEM) image of NiFe₂O₄/Sil/FA/Carbpt at a resolution of 50 μm, according to certain embodiments.

FIG. 5B is an elemental mapping image for the distribution of silicon (Si) at a resolution of 2.5 μm, according to certain embodiments.

FIG. 5C is an elemental mapping image for the distribution of oxygen (O) at a resolution of 2.5 μm, according to certain embodiments.

FIG. 5D is an elemental mapping image for the distribution of nickel (Ni) at a resolution of 2.5 μm, according to certain embodiments.

FIG. 5E is an elemental mapping image for the distribution of iron (Fe) at a resolution of 2.5 μm, according to certain embodiments.

FIG. 5F is an elemental mapping image for the distribution of platinum (Pt) at a resolution of 2.5 μm, according to certain embodiments.

FIG. 5G is an elemental mapping image for the distribution of carbon (C) at a resolution of 2.5 μm, according to certain embodiments.

FIG. 5H is an energy dispersive X-ray (EDX) spectra of NiFe₂O₄/Sil/FA/Carbpt, according to certain embodiments.

FIG. 5I shows a high-resolution transmission electron microscopy (HRTEM) image for NiFe₂O₄/Sil/FA/Carbpt at a resolution of 50 nm, according to certain embodiments.

FIG. 5J shows a HRTEM image of NiFe₂O₄/Sil/FA/Carbpt at a resolution scale of 10 nm, according to certain embodiments.

FIG. 6A is a graph depicting percentage (%) cumulative Carbpt release of Sil/Carbpt, NiFe₂O₄/Sil/Carbpt and NiFe₂O₄/Sil/FA/Carbpt at 5.6 pH using dialysis membrane technique, according to certain embodiments.

FIG. 6B is a graph depicting percentage cumulative Carbpt release of Sil/Carbpt, NiFe₂O₄/Sil/Carbpt and NiFe₂O₄/Sil/FA/Carbpt at 7.4 pH condition dialysis membrane technique, according to certain embodiments.

FIG. 6C is a graph depicting percentage cumulative Carbpt release of Sil/Carbpt, NiFe₂O₄/Sil/Carbpt and NiFe₂O₄/Sil/FA/Carbpt at 2.0 pH condition dialysis membrane technique, according to certain embodiments.

FIG. 6D is a graph depicting percentage cumulative Carbpt release of Sil/Carbpt, NiFe₂O₄/Sil/Carbpt and NiFe₂O₄/Sil/FA/Carbpt at 9.0 pH condition dialysis membrane technique, according to certain embodiments.

FIG. 7A shows a scanning transmission electron microscopy (STEM) image of NiFe₂O₄/Sil/FA/Carbpt, according to certain embodiments.

FIG. 7B shows a plot of the thermogravimetric analysis (TGA) of NiFe₂O₄/Sil/FA, according to certain embodiments.

FIG. 7C shows the Fourier transform infrared (FTIR) spectra of NiFe₂O₄/Sil, NiFe₂O₄/Sil/Carbpt and NiFe₂O₄/Sil/FA/Carbpt, according to certain embodiments.

FIG. 7D illustrates the Carbpt adsorption on NiFe₂O₄/Sil over a duration of from 0.25 hours (h) to 48 h, according to certain embodiments.

FIG. 7E depicts an ultraviolet (UV) absorbance spectrum for Carbpt and FA with a release time between 0.25 hours to 48 hours, according to certain embodiments.

FIG. 7F is an illustration of amide bond formation due to condensation between the amine group of Carbpt and the carbonyl group of FA, according to certain embodiments.

FIG. 7G is a schematic representation of the multifunctional NiFe₂O₄/Sil/FA/Carbpt smart selective drug delivery system for colon and cervical cancer treatment, according to certain embodiments.

FIG. 8 shows the mass magnetization (emu/g) properties of NiFe₂O₄/Sil nanocomposite with variance in magnetic field (Oe), according to certain embodiments.

FIG. 9 depicts a logarithmic dose-response curve showing cytotoxic activities of NiFe₂O₄/Sil, NiFe₂O₄/Sil/Carbpt, NiFe₂O₄/Sil/FA/Carbpt, and Sil/Cisplatin (Sil/Cispt) on HFF-1 cells, according to certain embodiments.

FIG. 10 depicts a logarithmic dose-response curve depicting cytotoxic activities of NiFe₂O₄/Sil, NiFe₂O₄/Sil/Carbpt, NiFe₂O₄/Sil/FA/Carbpt, and Sil/Cispt on HCT116 cells, according to certain embodiments.

FIG. 11 is a logarithmic dose-response curve showing cytotoxic activities of NiFe₂O₄/Sil, NiFe₂O₄/Sil/Carbpt, NiFe₂O₄/Sil/FA/Carbpt, and Sil/Cispt on HeLa cells, according to certain embodiments.

FIG. 12A is an image of the cell morphology HFF-1 cells treated for 24 hours with 40 micrograms per milliliter (μg/mL) of control, according to certain embodiments.

FIG. 12B is an image of the cell morphology of HFF-1 cells treated for 24 hours with 40 μg/mL of NiFe₂O₄/Sil/Carbpt, according to certain embodiments.

FIG. 12C is an image of the cell morphology of HFF-1 cells treated for 24 hours with 40 μg/mL of NiFe₂O₄/Sil/FA/Carbpt, according to certain embodiments.

FIG. 12D is an image of the cell morphology of HFF-1 cells treated for 24 hours with 40 μg/mL of NiFe₂O₄/Sil/Cispt, according to certain embodiments.

FIG. 12E is an image of the cell morphology of HFF-1 cells treated for 24 hours with 40 μg/mL of NiFe₂O₄/Sil, according to certain embodiments.

FIG. 12F is an image of the cell morphology of HFF-1 cells treated for 24 hours with 40 μg/mL of Cispt, according to certain embodiments.

FIG. 12G is an image of the cell morphology of HFF-1 cells treated for 24 hours with 40 μg/mL of Carbpt, according to certain embodiments.

FIG. 13A is an image of the cell morphology of HeLa cells treated for 24 hours with 20 μg/mL of control, according to certain embodiments.

FIG. 13B is an image of the cell morphology of HeLa cells treated for 24 hours with 20 μg/mL of NiFe₂O₄/Sil/Carbpt, according to certain embodiments.

FIG. 13C is an image of the cell morphology of HeLa cells treated for 24 hours with 20 μg/mL of NiFe₂O₄/Sil/FA/Carbpt, according to certain embodiments.

FIG. 13D is an image of the cell morphology of HeLa cells treated for 24 hours with 20 μg/mL of NiFe₂O₄/Sil/Cispt, according to certain embodiments.

FIG. 13E is an image of the cell morphology of HeLa cells treated for 24 hours with 20 μg/mL of NiFe₂O₄/Sil, according to certain embodiments.

FIG. 13F is an image of the cell morphology HeLa cells treated for 24 hours with 20 μg/mL of Cispt, according to certain embodiments.

FIG. 13G is an image of the cell morphology of HeLa cells treated for 24 hours with 20 μg/mL of Carbpt, according to certain embodiments.

FIG. 14A is an image of the cell morphology of HCT116 cells treated for 24 hours with 20 μg/mL of control, according to certain embodiments.

FIG. 14B is an image of the cell morphology of HCT116 cells treated for 24 hours with 20 μg/mL of NiFe₂O₄/Sil/Carbpt, according to certain embodiments.

FIG. 14C is an image of the cell morphology of HCT116 cells treated for 24 hours with 20 μg/mL of NiFe₂O₄/Sil/FA/Carbpt, according to certain embodiments.

FIG. 14D is an image of the cell morphology of HCT116 cells treated for 24 hours with 20 μg/mL of NiFe₂O₄/Sil/Cispt, according to certain embodiments.

FIG. 14E is an image of the cell morphology of HCT116 cells treated for 24 hours with 20 μg/mL of NiFe₂O₄/Sil, according to certain embodiments.

FIG. 14F is an image of the cell morphology of HCT116 cells treated for 24 hours with 20 μg/mL of Cispt, according to certain embodiments.

FIG. 14G is an image of the cell morphology of HCT116 cells treated for 24 hours with 20 μg/mL of Carbpt, according to certain embodiments.

### DETAILED DESCRIPTION

Embodiments of the present invention will now be described more fully hereinafter with reference to the accompanying drawings wherever applicable, in that some, but not all, embodiments of the disclosure are shown.

When describing the present disclosure, the terms used are to be construed in accordance with the following definitions, unless a context dictates otherwise.

As used herein, the words ‘a,’ ‘an’ and the like generally carry a meaning of ‘one or more,’ unless stated otherwise.

Furthermore, the terms ‘approximately,’ ‘approximate,’ ‘about,’ and similar terms generally refer to ranges that include the identified value within a margin of 20%, 10%, or preferably 5%, and any values therebetween.

When amounts, concentrations, dimensions and other parameters are expressed in the form of a range, a preferable range, an upper limit value, a lower limit value or preferable upper and limit values, it should be understood that any ranges obtainable by combining any upper limit or preferable value with any lower limit or preferable value are also specifically disclosed, irrespective of whether the obtained ranges are clearly mentioned in the context.

A weight percent of a component, unless specifically stated to the contrary, is based on the total weight of the formulation or composition in which the component is included. For example, if a particular element or component in a composition or article is said to have 5 wt. %, it is understood that this percentage is in relation to a total compositional percentage of 100%.

As used herein, the term ‘particle’ refers to a small object that acts as a whole unit with regard to its transport and properties. As used herein, ‘nanoparticles’-sometimes contracted herein to NPs-refers to particles having a particle size of 1 nanometer (nm) to 1000 nm.

As used herein, the term ‘particulate’ refers to small, discrete particles or components of a substance, often in a solid form, that can be suspended in a gas or liquid.

As used herein, the term ‘nanocomposite’ refers to a composite material in which at least one dimension of a component thereof is in the nanometer size scale (<100 nm). The nanocomposites are thus poly-phase solid materials made up of two or more nanomaterials. The term includes all types of multiphase solid material in which one of the phases has one, two, or three dimensions of less than 100 nm, or structures having nanoscale repeat distances between the different phases that make up the material.

Unless otherwise stated, the term “particle size” refers to the largest axis of the particle. In the case of a generally spherical particle, the largest axis is the diameter.

The term “median volume particle size” (Dv50), as used herein, refers to a particle size corresponding to 50% of the volume of the sampled particles being greater than and 50% of the volume of the sampled particles being smaller than the recited Dv50 value. Particle size is determined herein by Scanning Electron Microscopy.

As used herein, the term ‘porous’ refers to a material that contains tiny holes or voids (pores) within its structure. As used herein, the term ‘porosity’ refers to a measure of the void or vacant spaces within a material.

As used herein, the term ‘pore size’ may be thought of as the length or longest dimension of a pore opening.

As used herein, the term ‘median pore diameter’ refers to the pore size in a material at which half of the pores are smaller and half are larger. It is a statistical measure used to describe the distribution of pore sizes in a porous material.

As used herein, the term ‘pore volume’ refers to the total volume of the pores (void spaces) within a material. It is typically expressed in units such as cubic centimetres per gram (cm³/g) or millilitres per gram (mL/g).

As used herein, the term ‘Korsmeyer-Peppas equation’ is a mathematical model used to describe the release behavior of drugs or other substances from solid or semi-solid matrices.

The term “powder”, as used herein, means a composition that consists of finely dispersed solid particles that are free-flowing.

The term “dry” as used herein means comprising less than 5 wt. % of any compound or composition being in liquid form when measured at 25° C. under ambient conditions. For instance, the term “dry” includes comprising less than 3 wt. %, less than 2 wt. %, less than 1%, or even about 0% of said compound or composition being in liquid form when measured at 25° C. under ambient conditions. Exemplary such compounds or compositions include water, oils, organic solvents and other wetting agents.

As used herein, the term ‘X-ray diffraction’ or ‘XRD’ or ‘X-ray crystallography’ refers to basic technique for obtaining information on the atomic structure of crystalline materials used as a standard laboratory technique. Unless otherwise specified, the XRD shall include an analytical technique based on the diffraction of X-rays by matter, especially for crystalline materials.

As used herein, the term ‘Scanning Electron Microscopy’ or ‘SEM’ refers to a surface-imaging technique that produces images of a sample by scanning the sample with a focused beam of electrons. Unless otherwise specified, the SEM shall include all imaging techniques using electron beams for imaging.

As used herein, ‘comminuting’ refers to process of reducing the average size of solid materials into smaller particles, by crushing, grinding, cutting, vibrating, or other processes.

As used herein, the term ‘calcination’ refers to a thermal treatment process which is conducted in the absence of, or under a restricted supply of ambient oxygen. This is performed to remove impurities or volatile substances and/or to induce thermal decomposition or a change in the thermally treated material.

The term ‘dropwise’ as used herein means that one discrete drop or aliquot of a liquid, irrespective of its size or volume, is administered at a time. Discrete drops or aliquots are administered consecutively: they may be provided at regular intervals, at irregular intervals or both such intervals may be applied over the course of administration of the liquid. Further, the volume of an aliquot or drop may be independently determined and thus may be varied over the course of administration of the liquid. Exemplary devices for dropwise addition of liquids include syringes and columns.

As used herein, ‘magnetic materials’ refers to materials that are impacted by external electromagnetic fields in their surroundings.

Magnetic NiFe₂O₄ is a type of ceramic material composed of nickel (Ni) and iron (Fe) in the form of a spinel ferrite. It has magnetic properties due to the presence of iron ions, which can align their magnetic moments in response to an external magnetic field. This material exhibits strong ferrimagnetic properties, meaning the magnetic moments of its ions are aligned in opposite directions but with unequal magnitudes, resulting in a net magnetic moment.

As used herein, the term ‘superparamagnetic’ refers to a property of certain nanoparticles or materials, where they behave like a magnet only in the presence of an external magnetic field but do not retain any magnetization once the field is removed. This occurs due to the small size of the particles, typically in the nanometer range, which results in the magnetic moments of the particles being able to flip direction easily on account of thermal energy. Superparamagnetic materials do not exhibit permanent magnetism, unlike ferromagnetic or ferrimagnetic materials, but they show strong magnetic responsiveness when exposed to an external field.

As used herein, the term ‘buffer solution’ is a solution that resists changes in its pH when small amounts of an acid or base are added. It typically consists of a weak acid and its conjugate base or a weak base and its conjugate acid.

As used herein, the term ‘cancer’ refers to all types of cancer, neoplasm or malignant tumors found in mammals, including leukemias, lymphomas, carcinomas, and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the terms ‘treat,’ ‘treatment,’ and ‘treating’ in the context of the administration of a therapy to a subject in need thereof refer to the reduction or inhibition of the progression and or duration of cancer, the reduction or amelioration of the severity of cancer, and/or the amelioration of one or more symptoms thereof resulting from the administration of one or more therapies. In some embodiments, the subject is a mammalian subject. In one embodiment, the subject is a human. ‘Treating’ or ‘treatment’ of a disease includes preventing the disease from occurring in a subject that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to cancer or hyperplasia, these terms simply mean that the life expectancy of an individual affected with cancer will be increased or that one or more of the symptoms of the disease will be reduced. In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%; and, (12) an increase in the number of patients in remission. In certain embodiments, such terms refer to a stabilization or reduction in cancer stem cell population. In some embodiments, such terms refer to a stabilization or reduction in the growth of cancer cells. In some embodiments, such terms refer to stabilization or reduction in cancer stem cell population and a reduction in the cancer cell population. In some embodiments, such terms refer to a stabilization or reduction in the growth and or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in the survival rate of a patient population. In further embodiments, such terms refer to an increase in the response rate, the durability of response, or the number of patients who respond or are in remission. In some embodiments, such terms refer to a decrease in the hospitalization rate of a patient population and/or a decrease in hospitalization length for a patient population.

As used herein, cytotoxicity in cell culture is typically expressed in terms of “LC₅₀” which denotes that concentration of the given active agent which is lethal to 50% of the targeted cells.

As used herein, the term ‘pharmaceutical composition’ refers to a mixture of the compounds described herein or pharmaceutically acceptable salts, esters, or prodrugs thereof, with other chemical components, such as physiologically acceptable carriers and excipients.

As used herein, the term ‘excipient’ refers to an inert substance added to a pharmaceutical composition to facilitate the administration of a compound further. Examples, without limitation, of excipients include calcium carbonate, calcium phosphate, various sugars and types of starch, cellulose derivatives, gelatin, vegetable oils, and polyethylene glycols.

As used herein, the term ‘administering’ means oral administration, administration as a suppository, topical contact, intravenous, parenteral, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal, of which examples include buccal, sublingual, palatal, gingival, nasal, vaginal, rectal, or transdermal administration. Parenteral administration includes, for instance, intravenous, intramuscular, intra-arteriole, intradermal, subcutaneous, intraperitoneal, intraventricular, and intracranial. Other modes of delivery include, but are not limited to, the use of liposomal formulations, intravenous infusion, and transdermal patches. It is not precluded in the present disclosure that administration may not include administration of any active agent other than the recited active agent. In some embodiments, the administration of the anticancer compound is selected from a group including intravenous, interperitoneal, intramuscular, and oral administration.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopes of naturally occurring nickel ²⁸Ni include ⁵⁸Ni, ⁶⁰Ni, ⁶¹Ni, ⁶²Ni, and ⁶⁴Ni. Isotopes of iron include ⁵⁴Fe, ⁵⁶Fe, ⁵⁷Fe, and ⁵⁸Fe. Isotopically-labeled compounds of the disclosure may generally be prepared by conventional techniques known to those skilled in the art or by processes analogous to those described herein, using an appropriate isotopically-labeled reagent in place of the non-labeled reagent otherwise employed.

Aspects of the present disclosure are directed to method of treating cancer cells to achieve apoptosis, comprising: contacting the cancer cells with a porous particulate nanocomposite in an amount sufficient to kill the cancer cells. The porous particulate nanocomposite comprises: a magnetic nickel ferrite (NiFe₂O₄); and, monodisperse spherical silica particles onto which the magnetic nickel ferrite (NiFe₂O₄) is dispersed, wherein the nanocomposite is functionalized with cis-diammine(cyclobutane-1,1-dicarboxylate-O,O′)platinum(II) (carboplatin) and folic acid, and further wherein the magnetic nickel ferrite (NiFe₂O₄) has an inverse spinel crystal structure. The cancer cells are cells of a cancer selected from the group consisting of colon cancer, colorectal cancer and cervical cancer.

This study developed a pH-sensitive, magnetic, and folate-based drug delivery system for targeted Carbpt release, showing selective efficacy for cervical, colorectal and colon cancer while minimizing toxicity to normal cells.

A porous particulate nanocomposite is described. The nanocomposite comprises: a magnetic nickel ferrite (NiFe₂O₄); and, monodisperse spherical Sil particles onto which the magnetic NiFe₂O₄ is dispersed. The nanocomposite exhibits enhanced magnetic properties, controlled particle size and distribution, and improved chemical stability due to the inclusion of Sil. The Sil matrix further contributes to the mechanical strength and thermal stability of the composite. Additionally, the surface of the Sil particles can be functionalized for specific applications. In an embodiment, the magnetic NiFe₂O₄ is deposited partially or wholly onto monodisperse spherical Sil particles in a uniform and continuous manner. In some embodiments, the magnetic NiFe₂O₄ forms a continuous layer onto the monodisperse spherical Sil particles. In an embodiment, the magnetic NiFe₂O₄ forms a monolayer onto the monodisperse spherical Sil particles. In another embodiment, the magnetic NiFe₂O₄ may include more than a single layer onto the monodisperse spherical Sil particles.

The nanocomposite is functionalized with cis-diammine(cyclobutane-1,1-dicarboxylate-O,O′)platinum(II) (carboplatin) (hereafter referred to as Carbpt) and folic acid (FA). In some embodiments, FA is present in an amount of from about 1 to about 10 wt. %, based on the weight of the nanocomposite. For example, the folic acid may be present in an amount of from about 2 to about 10 wt. % or from about 2 to about 8 wt. %, based on the weight of the nanocomposite.

The inclusion of Carbpt imparts anti-cancer properties, enabling targeted therapeutic application, while FA facilitates selective binding to folate receptors, enhancing the specificity and efficiency of drug delivery to cancer cells. Additionally, alternative functionalizing compounds, such as amino acids, antibodies, peptides, or polymers, may be employed to achieve desired functionalities, including improved biocompatibility, controlled drug release, or enhanced imaging capabilities. In some embodiments, the FA is present in an amount of from about 1-10 wt. %, for example about 2-9 wt. %, about 3-8 wt. %, about 4-7 wt. %, or about 5-6 wt. %, based on the weight of the nanocomposite. In some embodiments, FA is present in an amount of about 3-7 wt. %, about 3.5-6.5 wt. %, about 4-6 wt. %, or about 4.5-5.5 wt. %, based on the weight of the nanocomposite. In some embodiments, the Carbpt is present in an amount of about 1-10 wt. %, for example about 2-9 wt. %, about 3-8 wt. %, about 4-7 wt. %, or about 5-6 wt. %, based on the weight of the nanocomposite. In some embodiments, Carbpt is present in an amount of about 3-7 wt. %, for example about 3.5-6.5 wt. %, about 4-6 wt. %, or about 4.5-5.5 wt. %, based on the weight of the nanocomposite.

The magnetic NiFe₂O₄ has an inverse spinel crystal structure, which imparts enhanced magnetic properties, improved chemical and thermal stability, and a high surface area. Spinel oxides having AB₂O₄ (A=Mn, Cu, Co, Zn, Fe, Ni; B=Cr, Ni, Mn, Mo, Co) formula have normal, inverse, or complex structures determined by cation occupation of octahedral (Oh) or tetrahedral (Td) sites. The unit cell consists of a face-centered cubic arrangement of oxygen ions, containing 32 O²⁻ ions, with 64 tetrahedral and 32 octahedral sites occupied by A²⁺ and B³⁺ cations. When one half of the octahedral interstices are occupied by B³⁺ cations and one-eighth of the tetrahedral sites are occupied by A²⁺ cations, the spinel phase is termed “normal”. Conversely, when tetrahedral sites are occupied by half of the B³⁺ and octahedral sites by the other half along with A²⁺, the spinel phase is termed “inverse”.

The NiFe₂O₄ is superparamagnetic. The superparamagnetic properties of the NiFe₂O₄ in the nanocomposite provide enhanced magnetic responsiveness, preventing aggregation and ensuring stability in biological environments.

In some embodiments, the nanocomposite is porous. A porous material is the one that forms a porous bulk solid. Pores may be micropores, mesopores, macropores, and/or a combination thereof. The pores exist in the bulk material, not necessarily in the molecular structure of the material. The term ‘microporous’ means that nanocomposite have pores with an average pore width (i.e. diameter) of less than 2 nm. The term ‘mesoporous’ means the pores of the nanocomposite have an average pore width of 2-50 nm. The term ‘macroporous’ means the pores of nanocomposite have an average pore width larger than 50 nm. Pore size may be determined by methods including, but not limited to, gas adsorption (e.g. N₂ adsorption), mercury intrusion porosimetry, and imaging techniques such as scanning electron microscopy (SEM), and X-ray computed tomography (XRCT).

The Brunauer Emmett Teller (BET) hypothesis is the foundation for a significant analysis method for determining the specific surface area of a material. It attempts to explain the physical adsorption of gas molecules on a solid surface. Specific surface area is a property of solids, which is the total surface area of a material per unit of mass, solid or bulk volume, or cross-sectional area. In some embodiments, pore diameter, pore volume, and BET surface area are measured by gas adsorption analysis, preferably N₂ adsorption analysis (e.g., N₂ adsorption isotherms).

In some embodiments, the functionalized nanocomposite has a surface area of from about 30-50 square meter per gram (m²/g), for example about 31-49 m²/g, about 32-48 m²/g, about 33-47 m²/g, about 36-46 m²/g, about 37-45 m²/g, about 38-44 m²/g, about 39-43 m²/g, or about 40-42 m²/g, as determined by BET analysis. In some embodiments, the functionalized nanocomposite has a surface area of from about 30-40 m²/g, for example about 31-39 m²/g, about 32-38 m²/g, about 33-37 m²/g, or about 34-36 m²/g, as determined by BET analysis. In a preferred embodiment, the functionalized nanocomposite has a surface area of 35 m²/g, as determined by BET Analysis

In some embodiments, the pore distribution of the nanocomposite may include, but is not limited to, unimodal, bimodal, trimodal, multimodal, narrow, broad, or Gaussian pore size distributions. In a preferred embodiment, the average pore distribution of nanocomposite is unimodal, indicating a single dominant pore size within the material. This unimodal distribution suggests that the nanocomposites possess a relatively narrow pore size range, resulting in more uniform pore structures and enhancing specific properties such as adsorption capacity, catalytic activity, or surface reactivity.

In some embodiments, the functionalized nanocomposite has a median pore diameter of from about 10-20 nm, for example about 11-19 nm, about 12-18 nm, about 13-17 nm, or about 14-16 nm, as determined by Barrett-Joyner-Halenda (BJH) desorption analysis. In some embodiments, the functionalized nanocomposite has a median pore diameter of about 15-20 nm, for example of about 16-19 nm, or preferably about 17-18 nm, as determined by BJH desorption analysis. In a preferred embodiment, the functionalized nanocomposite has a median pore diameter of 16.5 nm.

In some embodiments, the functionalized nanocomposite has a pore volume of about 0.1-0.3 cm³/g, preferably about 0.15-0.25 cm³/g, as determined by BJH desorption analysis. In some embodiments, the functionalized nanocomposite has a pore volume of about 0.1-0.2 cm³/g, for example about 0.11-0.19 cm³/g, about 0.12-0.18 cm³/g, about 0.13-0.17 cm³/g, or preferably about 0.14-0.16 cm³/g, as determined by BJH desorption analysis. In a preferred embodiment, the functionalized nanocomposite has a pore volume of 0.15 cm³/g.

The functionalized nanocomposite includes amide bonds, as determined by Fourier Transform Infrared Spectroscopy (FTIR). The amide bonds in the functionalized nanocomposite enhances its stability, biocompatibility, and molecular design flexibility. These bonds enable efficient functionalization for linking bioactive molecules or therapeutic agents. The FTIR spectrum of the functionalized nanocomposite has also presents an Si—OH peak of lower intensity than that of an FTIR spectrum of a porous particulate nanocomposite of magnetic NiFe₂O₄ and monodisperse spherical Sil particles which is functionalized only with FA.

The Korsmeyer-Peppas equation is Mₜ/M_{∞}=k.tⁿ in which: Mₜ/M_{∞} represents the fraction of Carbpt released from the nanocomposite at time, t. In some embodiments, the functionalized nanocomposite has, in a phosphate buffered solution (PBS) at a pH of about 7.4, a release constant k of from about 5 to about 30 h⁻ⁿ, for example about 10 to about 25 h⁻ⁿ, about 15 to about 20 h⁻ⁿ, or about 16.5960±2.2181; and, a transport exponent n of from about 0.05 to about 0.30, for example about 0.10 to about 0.25, or about 0.15 to about 0.20. In some embodiments, the functionalized nanocomposite has, in a phosphate buffered solution (PBS) at a pH of about 7.4, a release constant k of from about 10 to about 20 h⁻ⁿ, for example about 12 to about 18 h⁻ⁿ, about 14 to about 16 h⁻ⁿ, or, preferably 16.5960±2.2181; and, a transport exponent n of from about 0.05 to about 0.15, for example about 0.75 to about 0.125, or, preferably 0.1031±0.0569.

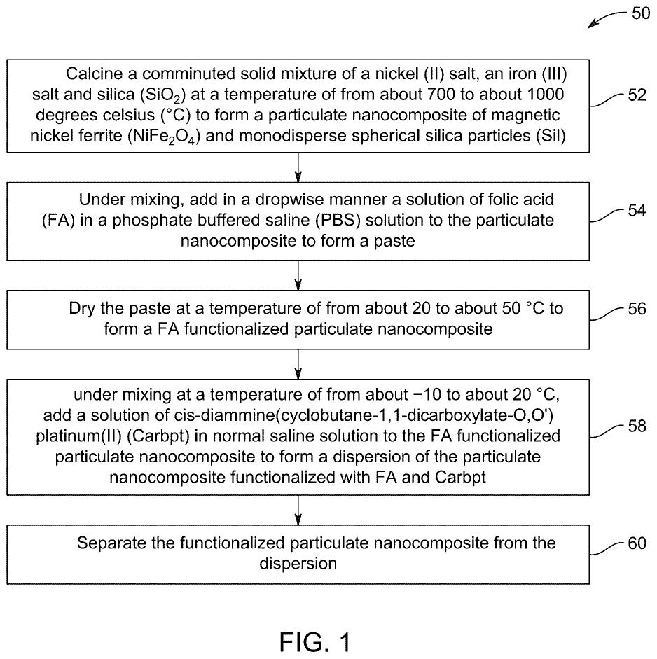

FIG. 1 illustrates a flow chart of a method 50 of preparing the functionalized nanocomposite. The order in which the method 50 is described is not intended to be construed as a limitation, and any number of the described method steps can be combined in any order to implement the method 50. Additionally, individual steps may be removed or skipped from the method 50 without departing from the spirit and scope of the present disclosure.

At step 52, the method 50 includes calcining a comminuted solid mixture of a nickel (II) salt, an iron (III) salt and Sil at a temperature of from about 700-1000° C., for example about 710-990° C., about 720-980° C., about 730-970° C., about 740-960° C., about 750-950° C., about 760-940° C., about 770-930° C., about 780-920° C., about 790-910° C., about 800-900° C., about 810-890° C., about 820-880° C., about 830-870° C., or about 840-860° C. to form a particulate nanocomposite of magnetic NiFe₂O₄ and monodisperse spherical Sil particles. In a preferred embodiment, the method includes calcining the comminuted solid mixture of a nickel (II) salt, an iron (III) salt and Sil at a temperature of 850° C.

The calcination is carried out by heating it to a high temperature under a restricted supply of ambient oxygen. This is performed to remove impurities or volatile substances and to incur thermal decomposition. Typically, the calcination is carried out in a furnace preferably equipped with a temperature control system, which may provide a heating rate of up to 50 degrees Celsius per minute (° C./min), for instance up to 40° C./min, up to 30° C./min, up to 20° C./min, up to 10° C./min, up to 5° C./min, up to 2° C./min, or preferably up to 1° C./min. In a preferred embodiment, the calcination includes a heating rate of 5° C./min.

The nickel (II) salt is desirably selected from the group consisting of nickel sulfate (NiSO₄), nickel nitrate (Ni(NO₃)₂), nickel chloride (NiCl₂) and nickel acetate (Ni(CH₃COO)₂). Other nickel salts that may be used to form the nanocomposite may include, but are not limited to, nickel carbonate, nickel bromide, nickel hydroxide, nickel phosphate, nickel sulfate, nickel citrate, nickel iodide, nickel chloride, nickel perchlorate, nickel nitrate, nickel triflate, nickel bis (trifluoromethanesulfonyl) imide, nickel tetrafluoroborate, and/or its hydrate, or mixtures thereof.

The iron (III) salt is desirably selected from the group including iron sulfate (Fe₂(SO₄)₃), iron nitrate (Fe(NO₃)₃), iron chloride (FeCl₃) and iron acetate (Fe(CH₃COO)₃). Other iron salts that may be used to form the nanocomposite may include iron bromide, iron sulfate, iron phosphate, iron hydroxide, iron phosphate tetrahydrate, iron chloride hydrate, iron chloride tetrahydrate, iron fluoride, ammonium iron sulfate hexahydrate, iron citrate tribasic monohydrate, iron gluconate dehydrate, iron pyrophosphate, iron phthalocyanine, iron phthalocyanine chloride, ammonium iron citrate, ammonium iron sulfate, iron chloride, iron bromide, iron chloride hexahydrate, ferric citrate, iron fluoride, iron nitrate nonahydrate, iron gluconate hydrate, iron iodide, iron lactate hydrate, iron oxalate dehydrate, ferrous sulfate heptahydrate, iron sulfide, iron acetate, iron fluoride tetrahydrate, iron iodide tetrahydrate, iron perchlorate hydrate, iron acetylacetonate, iron acetylacetonate, and iron ascorbate or its hydrate, or mixtures thereof.

In a preferred embodiment, the nickel salt is nickel nitrate and iron salt are iron nitrate. At step 54, the method 50 includes adding in a dropwise manner a solution of FA in a phosphate buffered saline solution under mixing to the particulate nanocomposite to form a paste. In some embodiments, the mixing can be done by stirring, swirling, sonicating, or a combination thereof may be employed to form the paste.

At step 56, the method 50 includes drying the paste at a temperature of from about 20-50° C., for example about 21-49° C., about 22-48° C., about 23-47° C., about 24-46° C., about 25-45° C., about 26-44° C., about 27-43° C., about 28-42° C., about 29-41° C., about 30-40° C., about 31-39° C., about 32-38° C., about 33-37° C., or about 34-36° C., to form a FA functionalized particulate nanocomposite. In some embodiments, the paste may be dried by using heating appliances such as ovens, microwaves, autoclaves, hot plates, heating mantles and tapes, oil baths, salt baths, sand baths, air baths, hot-tube furnaces, and hot-air guns. In a preferred embodiment, the paste is dried at room temperature.

At step 58, the method 50 includes adding a solution of Carbpt in normal saline solution to the FA functionalized particulate nanocomposite under mixing at a temperature of from about −10 to about 20° C., for example from about −8 to about 18° C., from about −6 to about 16° C., from about −4 to about 14° C., from about −2 to about 12° C., from about 0 to about 10° C., from about 2 to about 8° C., or from about 4 to about 6° C. to form a dispersion of the particulate nanocomposite functionalized with FA and Carbpt.

At step 60, the method 50 includes separating the functionalized particulate nanocomposite from the dispersion. The separation may be done by filtration, centrifugation, decantation, dialysis, and magnetic separation. In a preferred embodiment, the separation is done by filtration.

The present disclosure provides a method of treating cancer cells to achieve apoptosis comprising: contacting the cancer cells with the functionalized porous particulate nanocomposite as described above in an amount sufficient to kill the cancer cells. The cancer cells are cells of a cancer selected from the group consisting of colon cancer, colorectal cancer and cervical cancer.

The present disclosure also provides a method of providing treatment of at least one condition selected from the group including colon cancer, colorectal cancer and cervical cancer comprising administering the functionalized nanocomposite as described herein to a subject in need of treatment.

There is also provided a pharmaceutical composition comprising the functionalized nanocomposite as described herein and at least one pharmaceutically acceptable excipient. The pharmaceutical composition of the present disclosure can be administered orally, systemically, parenterally, by inhalation spray, rectally, or topically in dosage unit formulations containing conventional non-toxic pharmaceutically acceptable carriers, adjuvants, and vehicles as desired. In some embodiments, the method of administration of the pharmaceutical composition is oral. In other embodiments, the pharmaceutical composition is administered by injection, such as, for example, through a peritumoral injection.

Topical administration can also involve the use of transdermal administration, such as transdermal patches or iontophoresis devices. The term parenteral, as used herein, includes intravesical, intradermal, transdermal, subcutaneous, intramuscular, intralesional, intracranial, intrapulmonary, intracardial, intrasternal, and sublingual injections, or infusion techniques.

Injectable preparations, such as sterile injectable aqueous or oleaginous suspensions, can be formulated according to the known art using suitable dispersing or wetting agents and suspending agents. The sterile injectable preparation can also be a sterile injectable solution or suspension in a nontoxic, parenterally acceptable diluent or solvent, such as 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose, any fixed oil can be employed, including synthetic mono-or diglycerides. In addition, fatty acids such as oleic acid find use in the preparation of injectables. Dimethyl acetamide, surfactants including ionic and non-ionic detergents, and polyethylene glycols can be used. Mixtures of solvents and wetting agents, such as those discussed above, are also useful. Suppositories for rectal administration of the pharmaceutical composition can be prepared by mixing the functionalized nanocomposite with a suitable non-irritating excipient such as cocoa butter, synthetic mono-di-or triglycerides, fatty acids, and polyethylene glycols that are solid at ordinary temperatures but liquid at the rectal temperature and will therefore melt in the rectum and release the drug.

Solid dosage forms for oral administration can include capsules, tablets, pills, powders, and granules. In such solid dosage forms, the compounds of this disclosure are ordinarily combined with one or more adjuvants appropriate to the indicated route of administration. If administered in a solid dosage form, a functionalized nanocomposite can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the functionalized nanocomposite in hydroxypropyl methylcellulose. In the case of capsules, tablets, and pills, the dosage forms can also include buffering agents, such as sodium citrate, magnesium or calcium carbonate, or bicarbonate. Tablets and pills can additionally be prepared with enteric coatings.

For therapeutic purposes, formulations for parenteral administration can be in the form of aqueous or non-aqueous isotonic sterile injection suspensions. These suspensions can be prepared from sterile powders or granules having one or more of the carriers or diluents mentioned for use in the formulations for oral administration. A contemplated functionalized nanocomposite of the present disclosure can be dispersed in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also include adjuvants, such as wetting agents, emulsifying and suspending, agents, and sweetening, flavouring, and perfuming agents.

In certain embodiments, the pharmaceutical composition of the present disclosure may be used in combination with one or more other antineoplastic or chemotherapeutic agents.

Exemplary chemotherapeutic agents include but are not limited to aflibercept, asparaginase, bleomycin, busulfan, carmustine, chlorambucil, cladribine, cyclophosphamide, cytarabine, dacarbazine, daunorubicin, doxorubicin, etoposide, fludarabine, gemcitabine, hydroxyurea, idarubicin, ifosamide, irinotecan, lomustine, mechclorethamine, melphalan, mercaptopurine, methotrexate, mitomycin, mitoxantrone, pentostatin, procarbazine, 6-thioguanine, topotecan, vinblastine, vincristine, retinoic acid, oxaliplatin, cis-platin, Carbpt, 5-FU (5-fluorouracil), teniposide, amasacrine, docetaxel, paclitaxel, vinorelbine, bortezomib, clofarabine, capecitabine, actinomycin D, epirubicine, vindesine, methotrexate, tioguanine (6-thioguaniue), tipifarnib. Examples of antineoplastic agents which are protein kinase inhibitors include imatinib, erlotinib, sorafenib, sunitinib, dasatinib, nilotinib, lapatinib, gefitinib, temsirolimus, everolimus, rapamycin, bosutinib, pazopanib, axitinib, neratinib, vatalanib, pazopanib, midostaurin, and enzastaurin. Exemplary antineoplastic agents which are antibodies include trastuzumab, cetuximab, panitumumab, rituximab, bevacizumab, mapatumumab, conatumumab, and lexatumumab.

### EXAMPLES

The following examples demonstrate a method of synthesizing a folic acid (FA) and cis-diammine(cyclobutane-1,1-dicarboxylate-O,O′)platinum(II) (carboplatin or Carbpt) functionalized nickel ferrite (NiFe₂O₄)/monodispersed spherical silica (Sil) porous particulate nanocomposite (NiFe₂O₄/Sil/FA/Carbpt) for cancer treatment. The examples are provided solely for illustration and are not to be construed as limitations of the present disclosure, as many variations thereof are possible without departing from the spirit and scope of the present disclosure.

### Example 1

### Materials and Methods

In the present disclosure, cell culture treatment was conducted using a previously established culture method [See: Yi, X. et al., A step-by-step multiple stimuli-responsive metal-phenolic network prodrug nanoparticles for chemotherapy, Nano Research, 15: 1205-1212 (2022), the disclosure of which is incorporated herein by reference in its entirety]: colorectal cancer (HCT116; ATCC® CCL-247™), cervical cancer (HELA; ATCC® CCL-2™) and human foreskin fibroblast (HFF-1; ATCC® SCRC-1041™) cells were independently grown in complete Dulbecco's Modified Eagle Medium (DMEM) at 37° C.° and in a 5 percent (%) carbon dioxide (CO₂) environment. A 96 well plate (Thermo Fisher, Waltham, MA, USA) were seeded with 15×10³ cells/well. At 75% cell coverage of the wells, 2.5 micro gram per milliliters (μg/mL), 5 μg/mL, 20 μg/mL and 40 μg/mL of ND220, ND265, ND126cp, ND126, carboplatin (hereinafter referred to as Carbpt) were added over the cells for 24 hours (h). Then a 3-(4,5-dimethylthiazol-2-yl)-2,5-diphenyltetrazolium bromide (MTT) cell survival assay was used to estimate the relative growth inhibition compared to control cells.

MTT (3-(4, 5-Dimethylthiazol-2-yl)-2, 5-Diphenyltetrazolium Bromide) assay were used to measure the cell viability following a previously published method [See: Yi, X., A step-by-step multiple stimuli-responsive metal-phenolic network prodrug nanoparticles for chemotherapy. Nano Research, 15:1205-1212 (2022), the disclosure of which is incorporated herein by reference in its entirety]. Briefly, MTT was added to control and treated cells in the 96 well plate for 3 h at 37° C. after washing with phosphate buffered saline (PBS), the crystalized formazan dye was solubilized by isopropanol (C₃H₈O) and hydrochloric acid (HCl). The optical density (OD) of solubilized dye was measured at 570 nanometer (nm) using A 96 well plate reader (Tecan Infinite® 200 PRO, Männedorf, Switzerland). Each treatment was expressed as the percentage of cell survival compared to the control by comparing the measured OD values to the control.

A two-tailed t-test was performed to determine the statistical significance of the MTT cell viability assay data (P<0.05). The analysis was based on a minimum of three independent experiments conducted in triplicates.

### Example 2

Synthesis of NiFe₂O₄ Nanoparticles (NPs)

According to the present disclosure, for synthesizing nickel ferrite NPs, stoichiometric amounts of nickel nitrate and iron nitrate salts were completely dissolved in deionized water with continuous stirring. The solution was then heated at 95° C. for 45 minutes (min.). Subsequently, the pH of the solution was adjusted to 7 using 25% ammonia (NH3) solution. After thorough mixing, the solution was heated at 115° C. for 1 hour and further heated at 375° C. until the gel formed, burned, and was finally calcined at 500° C. for 4 hours to obtain nickel ferrite NPs.

### Example 3

Synthesis of NiFe₂O₄/Sil

To synthesize NiFe₂O₄/Sil, 0.74 grams (g) of nickel nitrate and 1.03 g of iron nitrate were weighed and added to 1.4 g of Sil (SiO2). The mixture was thoroughly blended using a mortar and pestle. The resulted material was then calcined at 850° C. for 6 hours, with a heating rate of 5degree Celsius per minute (° C./min).

### Example 4

Synthesis of NiFe₂O₄/Sil/FA/Carbpt

For the synthesis of NiFe₂O₄/Sil/FA/Carbpt, 50 mg of folic acid (FA) was dissolved in 5mL phosphate-buffered saline (PBS) at pH 7 and added dropwise to 1000 mg of NiFe₂O_{4/}Sil. After mixing the composite a dry paste was obtained, which was then dried at room temperature. FA loaded NiFe₂O₄/Sil was then functionalized with Carbpt at a drug to NiFe₂O₄/Sil/FA ratio of 0.05 in 10 mL of normal saline solution (NSS). The mixture was stirred overnight under ice-cooled conditions and subsequently filtered to obtain NiFe₂O₄/Sil/FA/Carbpt. The filtrate obtained after washing with 5 mL PBS solution was analyzed to determine the remaining Carbpt using diffuse reflectance spectroscopy (DRS) UV measurement.

### Example 5

### Characterization of Spinel Ferrite

The phase, texture, morphology, magnetic properties, and stability of the material were analyzed using X-ray diffraction (XRD), Rigaku Benchtop Miniflex XRD), Brunauer-Emmett-Teller surface area analysis (BET, Micromeritics ASAP 2020), diffuse reflectance ultraviolet-visible spectroscopy (UV-Vis), scanning electron microscopy with energy-dispersive X-ray spectroscopy (SEM-EDX), transmission electron microscopy (TEM), thermogravimetric analysis-differential thermal analysis (TGA-DTA), and vibrating sample magnetometry (VSM). Results

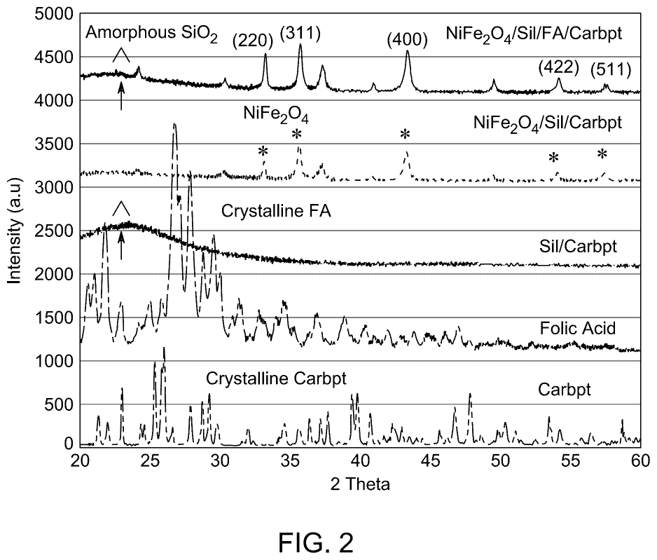

According to the present disclosure, folic acid (FA), a vital vitamin B ligand, has been utilized in targeted cancer therapy due to its ability to bind with cancer cells. The XRD analysis of Carbpt, FA, Sil/Carbpt, NiFe₂O₄/Sil/Carbpt and NiFe₂O₄/Sil/FA/Carbpt is shown in FIG. 2. The XRD pattern of FA alone exhibited the presence of several crystalline peaks between 20° to 50°, with peak maxima at 26.8. Similarly, Carbpt alone showed several crystalline peaks between 20° to 60°, as shown in FIG. 2. The loading of Carbpt on Sil showed only the presence of a broad amorphous Sil peak at 22.6° without the crystalline peaks of Carbpt. An analysis conducted by researchers has stated that such disappearance of crystal structure of Carbpt after loading on mesoporous Sil NPs (MSN) indicates a well dispersed drug interaction with hydroxyl groups without recrystallization inside the mesoporous cavities [See: Kargari Aghmiouni, D. et al., Dual-drug delivery by anisotropic and uniform hybrid nanostructures: A comparative study of the function and substrate-drug interaction properties, Pharmaceutics, 15 (4): 1214 (2023) the disclosure of which is incorporated herein in its entirety]. In the present disclosure, the nanocomposite formation of nickel ferrite with Sil showed several crystalline peaks indexed to (hkl) planes of (220), (311), (400), (422) and (511). In case of NiFe₂O₄/SS/FA/Carbpt, the crystalline peaks of both FA and Carbpt disappeared, indicating the nano transformations, while distinct spinel ferrite phases confirmed the presence of pure phase without any impurities. The Sil support also induces similar nanoformation of Carbpt, highlighting the specific role of the Sil nanocarrier in trans synthesizing the crystal properties of adsorbed Carbpt and FA.

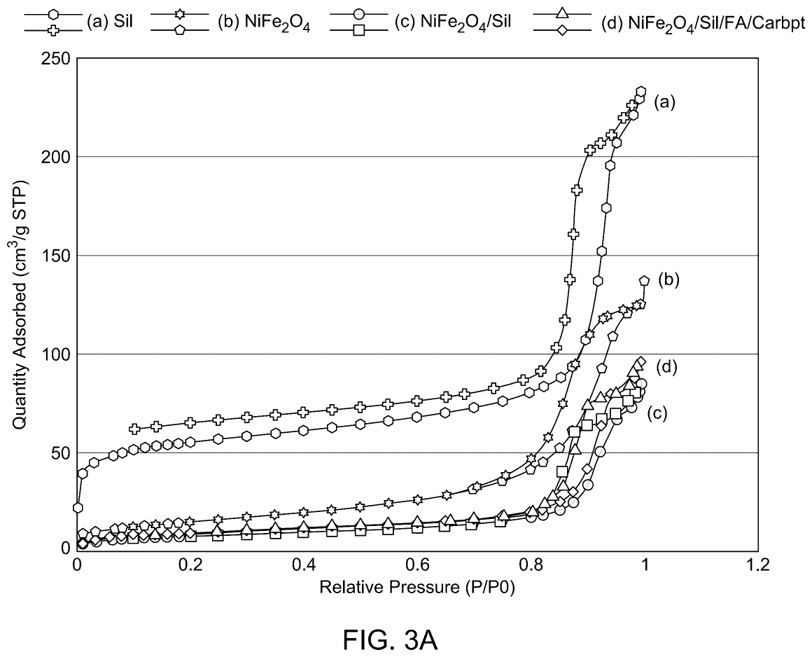

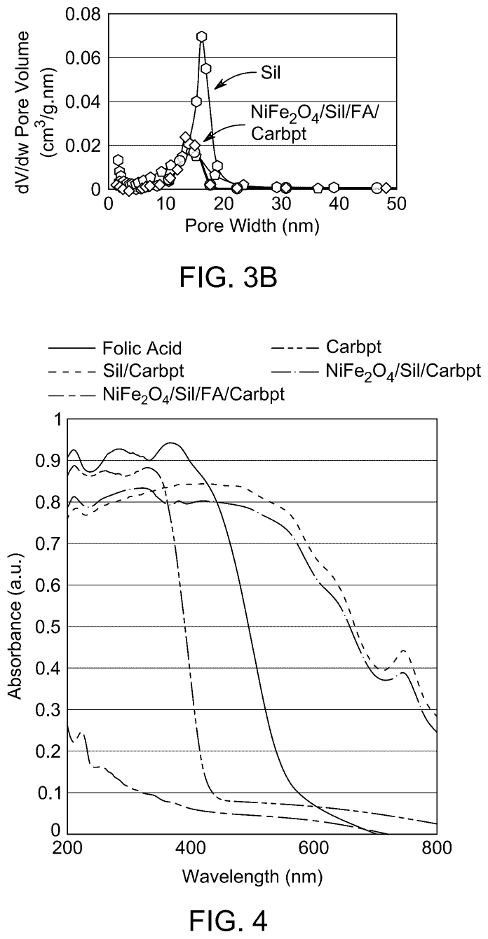

Nitrogen adsorption-desorption technique was used to determine the textural changes of nanoformulation after spinel ferrite, FA and Carbpt loading on Sil, as shown in FIGS. 3A-3B. Sil without modifications showed a type IV isotherm with H₂ hysteresis loop (FIG. 3A) indicating the mesopore formation with surface area of 170 meter square per gram (m²/g), as listed in Table 1. The pore size distribution (FIG. 3B) of Sil was uniform with average pore diameter of 8.3 nm and pore volume of 0.35 cm³/g. Nickel ferrite alone showed an innate mesoporous characteristic with surface area of 55 m²/g. In the case of the NiFe₂O₄/Sil nanocomposite formation, the surface area was measured to be 29 m²/g, indicating an 83% reduction compared to the initial value. The pore volume was 0.12 cm³/g, showing a 57% reduction, while the pore diameter increased to 17 nm, which represents approximately a two-fold increase. Such an increase in the pore diameter indicates the deposition of NiFe₂O₄ at the external pore surface of Sil and thereby contributing to the enlargement of external pore mouth. After FA and Carbpt loading on NiFe₂O₄/Sil, the surface area of NiFe₂O₄/Sil/FA/Carbpt was of 35 m²/g with pore volume of 0.15 cm³/g and pore diameter of 16.5 nm. The textural properties of the tested materials are summarized in Table 1 hereinbelow.

TABLE 1

| | BET surface | Pore volume | Average pore |
|---|---|---|---|
| NCs | area (m²/g) | (cm³/g) | size (nm) |
| NiFe₂O₄ | 55 | 0.19 | 14 |
| Sil | 170 | 0.35 | 8.3 |
| NiFe₂O₄/Sil | 29 | 0.12 | 17 |
| NiFe₂O₄/Sil/FA/Carbpt | 35 | 0.15 | 16.5 |

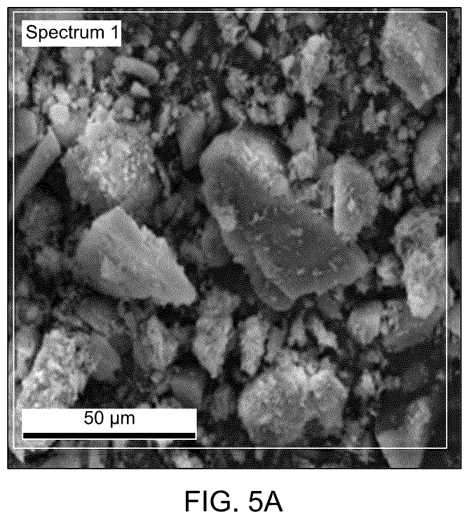

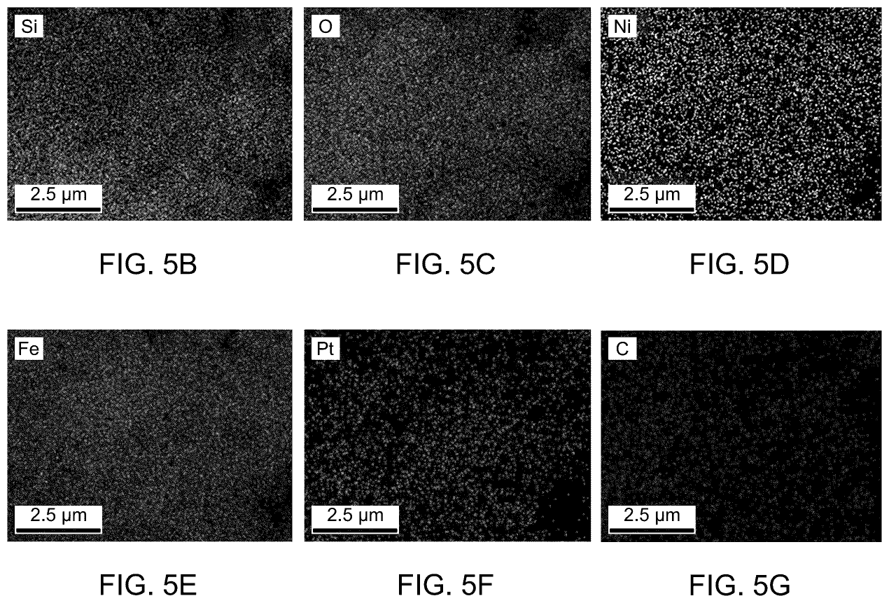

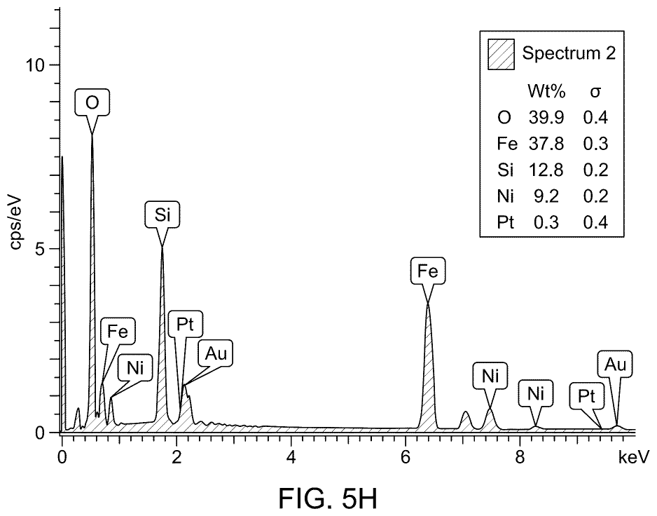

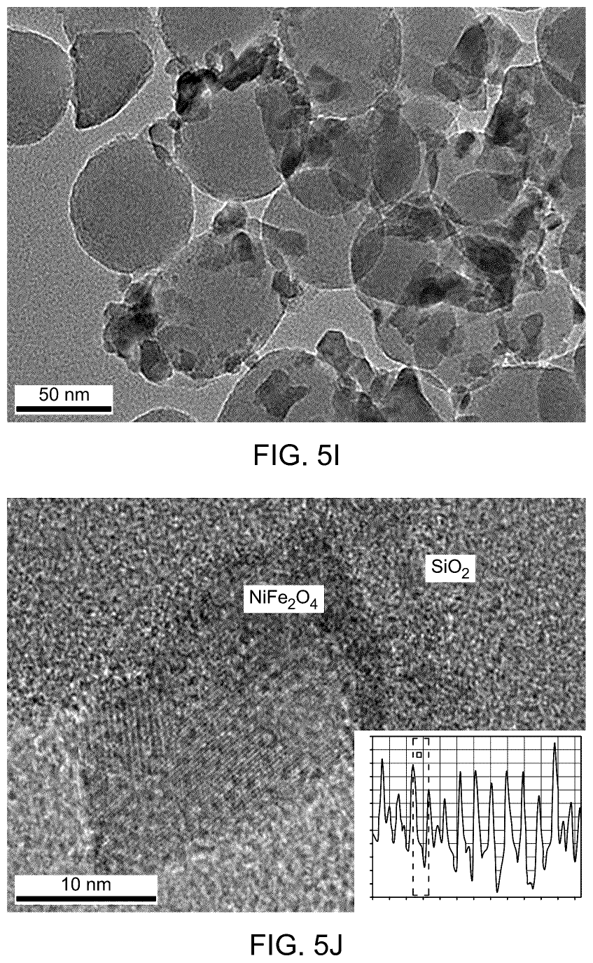

The chemical state variation of NiFe₂O₄, Carbpt, and FA on Sil was examined using Diffuse Reflectance Spectroscopy (DRS) spectroscopy. Carbpt and FA in free state showed a strong absorption at 280 nm and 370 nm, as shown in FIG. 4 [See: Lin, Q. et al, Environmentally friendly, one-pot synthesis of folic acid-decorated graphene oxide-based drug delivery system, Journal of Nanoparticle Research, 15:1-7 (2013), the disclosure of which is incorporated herein in its entirety]. Carbpt loading on Sil confirmed a strong absorption at about 220 nm indicating the presence of tetrahedral coordinated siloxane bonding in Sil. The coordination of Carbpt on Sil may be observed with extension of the absorption peaks corresponding to Carbpt at 260 nm and 340 nm, respectively. In case of NiFe₂O₄/Sil/Carbpt and NiFe₂O₄/Sil/FA/Carbpt, the characteristic peaks of Carbpt may be observed along with the peaks corresponding to nickel ferrite spinel that extends up to 700 nm, as shown in FIG. 4. This indicates the successful nanocomposite formation of nickel ferrite, FA and Carbpt on monodispersed Sil. FIG. 5A shows a SEM image of NiFe₂O₄/Sil/FA/Carbpt at a magnification of 50 μm. Mechanistic release of Carbpt (Carbpt) from FA-bound NiFe₂O₄/Sil nanocarrier, as shown in FIGS. 5B-5G depicts the SEM-EDX mapping of NiFe₂O₄/Sil/FA/Carbpt showing atomic distribution of silicon (Si), oxygen (O), nickel (Ni), iron (Fe), platinum (Pt), and carbon (C). FIG. 5H shows an EDX spectra of NiFe₂O₄/Sil/FA/Carbpt. FIGS. 5I-5J shows a HRTEM image for NiFe₂O₄/Sil/FA/Carbpt at a resolution of 50 nm and 10 nm, respectively.

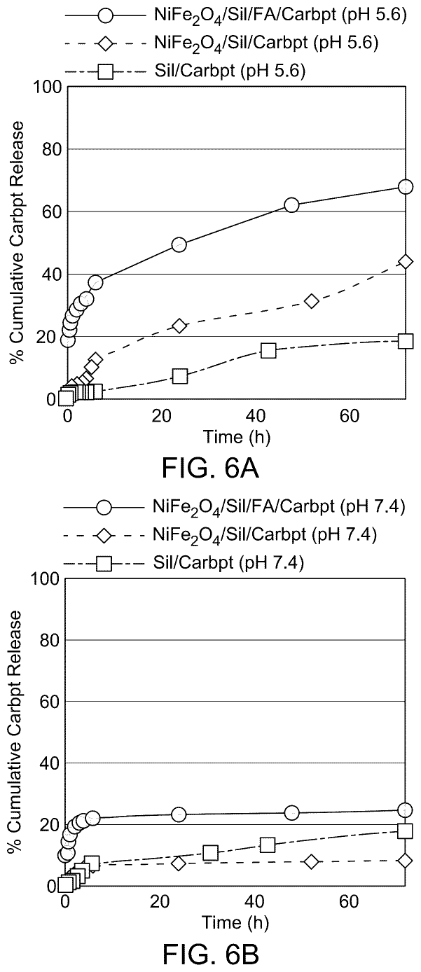

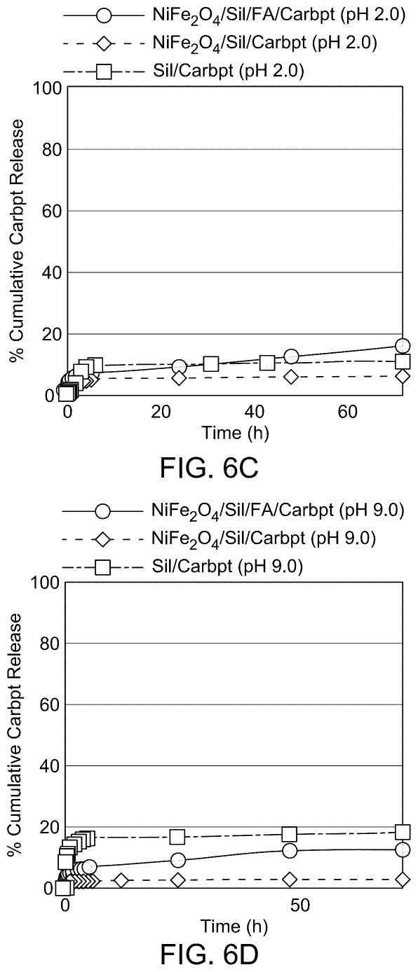

Carbpt has been widely used to treat ovarian cancer, small cell lung cancer, and squamous cell carcinomas. The anti-cancer efficacy of Carbpt is mainly due to its interaction with DNA, inhibiting cell proliferation. In the present disclosure, Carbpt was selected as a drug for the in vitro study of colon and cervical cancer, considering its lower toxicity and fewer side effects compared to cisplatin (Cispt). Carbpt encapsulation and loading capacity was 78% and 3.9%, respectively. The cervical cancer pH was about 5-6, colon cancer pH ranges from 7.4-9.0.Gastric intestinal pH was highly acidic between 1.2-2.0. After being internalized, the presence of FA increased the interaction with folate receptor and the pore opening lead to the drug release under various physiological environments. FA acted as a capping agent. To examine the pH based Carbpt release, in vitro release analysis was performed using three formulations Sil/Carbpt, NiFe₂O₄/Sil/Carbpt and NiFe₂O₄/Sil/FA/Carbpt at various pH conditions including 2.0° C., 5.6° C., 7.4° C., 9.0° C. at 37° C., as shown in FIGS. 6A-6D. At gastric pH 2.0, lower Carbpt release was observed for Sil/Carbpt, NiFe₂O₄/Sil/Carbpt, and NiFe₂O₄/Sil/FA/Carbpt, with releases of 11%, 6.3%, and 16% at 72 hours, respectively. In contrast, at cervical pH 5.6, higher Carbpt release was observed, with NiFe₂O₄/Sil/FA/Carbpt releasing 68%, NiFe₂O₄/Sil/Carbpt releasing 44%, and Sil/Carbpt releasing 19% at 72 hours. However, as the pH near the colon cancer pH condition, Carbpt release decreases to 25%, which shows the reduced disintegration and gate-keeper role of FA that safeguard the release of drug from the pores of Sil. However, such a slow drug release may be advantageous as it reduces the dose dependent Carbpt toxicity and permits use to be extended to a longer duration. After increasing the pH 9.0, a similar slow Carbpt release of 16% for 72 hours was observed that indicated the effective drug encapsulation without leakage. NiFe₂O₄/Sil/Cispt was used for comparative study.

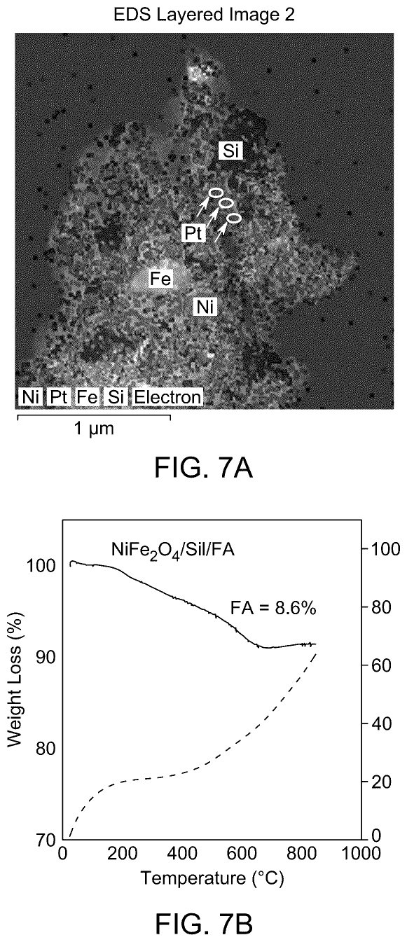

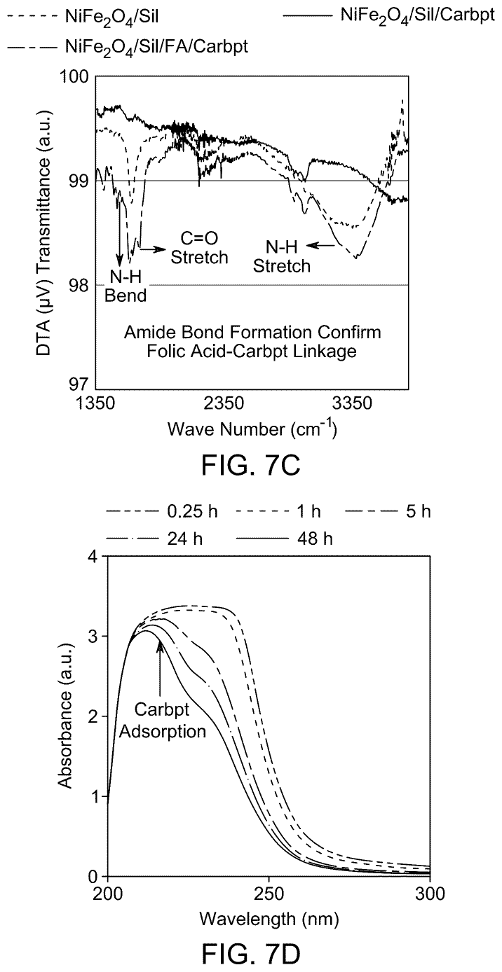

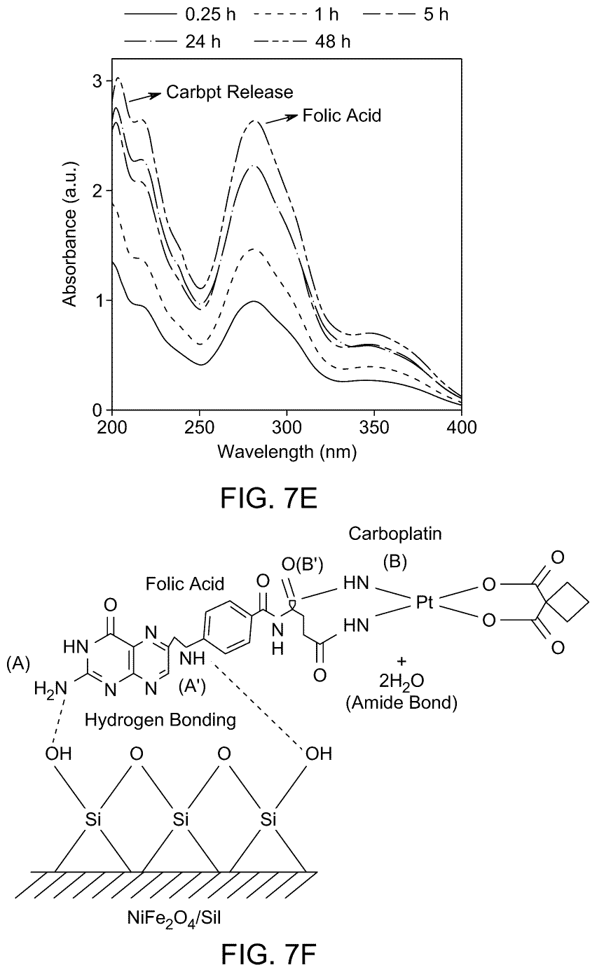

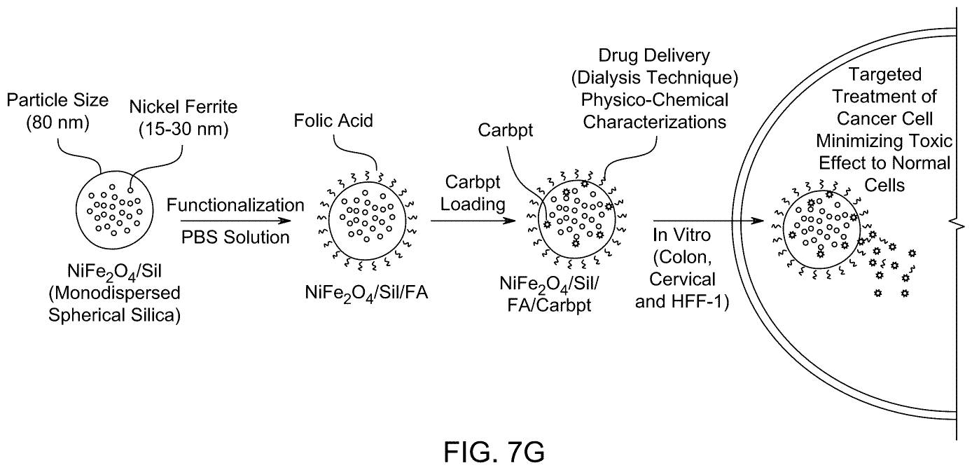

FIG. 7A shows the scanning transmission electron microscopy (STEM)-EDX mapping of the atomic distribution of Ni, Fe, Si and Pt of NiFe₂O₄/Sil/Carbpt. The analysis confirms the nickel ferrite cohabitation along with Pt of carboplatin on the silica matrix. The homogeneous distribution shows the advantage of the large surface area of the monodispersed silica and the presence of the four elements confirms the purity of NiFe₂O₄/Sil/Carbpt sample.

The thermogravimetric analysis (TGA) plot for NiFe₂O₄/Sil/FA is shown in FIG. 7B. TGA confirmed the interaction of FA functionalization with nanocarrier by providing information about the decomposition content from NiFe₂O₄/Sil/FA nanocarrier. At temperatures up to approximately 150° C., the decomposition curve indicated an initial weight loss corresponding to the evaporation of physically adsorbed water and dehydration of spherical Sil from the porous matrix. Folic acid (FA) underwent thermal disintegration between 250° C. and 350° C., resulting in an 8% weight loss to the sample. The extended temperature range of the weight loss demonstrated the thermal stability of FA within the Sil matrix.

The FTIR spectra of NiFe₂O₄/Sil, NiFe₂O₄/Sil/Carbpt and NiFe₂O₄/Sil/FA/Carbpt are shown in FIG. 7C. NiFe₂O₄/Sil exhibited sharp and broad elongated peaks at 1611 cm⁻¹ and 3334 cm⁻¹ corresponding to the hydroxyl O—H bending and Si—OH of Sil. NiFe₂O₄/Sil/Carbpt exhibited the Carbpt functional groups at 2905 cm⁻¹ and 2990 cm⁻¹. A reduction in the Si—OH peak of Sil indicated the interaction between Carbpt and Sil. In case of FA and Carbpt functionalization in the sample NiFe₂O₄/Sil/FA/Carbpt, additional peaks of carbonyl at 1684 cm⁻¹ and NH bending at 1520 cm⁻¹ confirmed the amide bond formation. Further, the absorption peak extended at 3325 cm⁻¹ indicating additional hydrogen and NH bond formation leading to new elongated stretching peaks.

Through Ultraviolet Spectral Analysis, the Carbpt adsorption effect on NiFe₂O₄/Sil was observed between the time duration of 0.25 hours to 48 hours, as shown in FIG. 7D. Carbpt exhibited a peak absorbance maximum at a wavelength of about 213 nm along with a shoulder peak at 231 nm. The presence of high textural characteristics tended to facilitate the pore accessibility of nanosized Carbpt leading to the reduction in Carbpt peaks, as listed in Table 1. After interaction with folic acid (FA) and Carbpt, the sample NiFe₂O₄/Sil/FA/Carbpt showed the additional band at about 285 nm confirming the interaction of FA with Carbpt. Furthermore, a systematic increase in the Carbpt and FA release was confirmed with the UV spectral analysis with a release time between 0.25 hours and 48 hours, as shown in FIG. 7E.

Folic acid (FA) has a bicyclic structure of pteridine ring, attached through methylene bridge (—CH₂—) with p-aminobenzoic acid followed by amide bond connecting glutamic acid containing functionally able carboxylic acid group. An analysis performed by Emtiazi G., et al. has reported the covalent FA conjugation with magnetic/diphenylalanine peptide derived nanotubes through carbodiimide coupling mechanism [See: Emtiazi, G. et al., Covalent diphenylalanine peptide nanotube conjugated to folic acid/magnetic nanoparticles for anti-cancer drug delivery, Journal of Drug Delivery Science and Technology, 41:90-98 (2017), the disclosure of which is incorporated herein in its entirety]. In the present case, NiFe₂O₄/Sil nanocarrier was composed of different types of silanol groups such as isolated silanol, vicinal and geminal, which facilitated the interaction with FA. Specifically, the presence of isolated hydroxyl group in silanol and terminal silanol groups in vicinal and geminal may have induced hydrophilic property that formed hydrogen bonds with amine functional group of FA (NH₂/NH) (A, A′).

FIG. 7F illustrates the mechanism of amide bond formation due to condensation between the amine group of Carbpt and the carbonyl group of FA. The drug Carbpt consists of a platinum atom attached with two amine groups (NH₃) (B) and two chelating ligands that formed bidentate cyclobutene-1,1-dicarboxylate ligand through carboxylate groups. During the nanocomposite formation between NiFe₂O₄/Sil, FA and Carbpt, hydrogen bonds were proposed between the silanol groups of NiFe₂O₄/Sil and the amine group of FA, while amide bond formation occurred due to condensation between the amine group of Carbpt and carbonyl group of FA (BB′), as shown in FIG. 7F. Due to the resonance characteristics of the amide bond, the bond became strong and rigid, similar to the structure of proteins that may withstand the acidic and basic medium of the colon and cervix. The presence of FA aided the selective interaction of NiFe₂O₄/Sil/FA/Carbpt with the receptor of cancer cells and reduced the toxic effect to normal cells, as shown in FIG. 7G.

The kinetics of Carbpt drug release were evaluated using the Korsmeyer-Peppas model. The Carbpt drug release profiles from nanocomposites at different pH were examined using the Korsmeyer-Peppas model, expressed using the equation.

$$R \% = k {t}^{n}$$

Where R % is the Carbpt drug percentage release at time (t), k and n are the kinetic rate constant and the release exponent, respectively. The kinetic parameters, together with their 95% confidence intervals, are listed in Table 2.

TABLE 2

| Carbpt drugs | k/h⁻ⁿ | n |
|---|---|---|
| NiFe₂O₄/Sil; pH 2.0 | 3.4216 + 0.2480 | 0.1637 + 0.0319 |
| NiFe₂O₄/Sil; pH 5.6 | 3.9773 + 0.4974 | 0.5424 + 0.0543 |
| NiFe₂O₄/Sil; pH 7.4 | 2.4405 + 0.9040 | 0.3634 + 0.1422 |
| NiFe₂O₄/Sil; pH 9.0 | 1.7026 + 0.0868 | 0.1535 + 0.0227 |
| NiFe₂O₄/Sil/FA; pH 2.0 | 4.9475 + 0.3176 | 0.2497 + 0.0284 |
| NiFe₂O₄/Sil/FA; pH 5.6 | 25.5458 + 0.9694 | 0.2177 + 0.0169 |
| NiFe₂O₄/Sil/FA; pH 7.4 | 16.5960 + 2.2181 | 0.1031 + 0.0569 |
| NiFe₂O₄/Sil/FA; pH 9.0 | 4.6009 + 0.4066 | 0.2466 + 0.0387 |
| Sil; pH 2.0 | 1.5775 + 1.2032 | 0.4979 + 0.1944 |
| Sil; pH 5.6 | 1.6971 + 0.4841 | 0.4841 + 0.0867 |
| Sil; pH 7.4 | 1.1197 + 0.4119 | 0.6166 + 0.1074 |
| Sil; pH 9.0 | 11.9033 + 1.2408 | 0.1254 + 0.0453 |

For NiFe₂O₄/Sil, the rate of Carbpt release, determined by the release constant, was enhanced at acidic and neutral pH. However, at pH 9.0, the rate of drug release was lower compared to the other pH values. Conversely, the release exponent (n) signified a fickian (0.16, 0.36, 0.15<0.45) and non-fickian (0.45<0.54<0.89) for pH values of 2.0, 7.4, 9.0 and 5.6, respectively. Similarly, for NiFe₂O₄/Sil/FA, the effect of pH on the rate of drug release followed the same pattern as NiFe₂O₄/Sil, with the highest rate of drug release recorded at weakly acidic condition, pH=5.6 and the lowest recorded at alkaline pH. The drug release mechanism followed the fickian diffusion mechanism at all pH conditions (n<0.45). In contrast, as regards the pH effect on the rate of release from Sil alone, Carbpt drug release followed an opposite trend to that of NiFe₂O₄/Sil and NiFe₂O₄/Sil/FA. Alkaline pH (pH=9.0) enhanced the drug release rate almost 10 times the acidic and neutral pH conditions. The release exponent (n) for the neutral (pH=7.4) and acidic (pH=2.0 and 5.6) signified a non-fickian diffusion mechanism while at alkaline pH (9.0), the drug release followed the fickian diffusion mechanism.

For the three nanoformulations described in Table 2, NiFe₂O₄/Sil/FA has the highest drug release rate at acidic and neutral pH, while Sil alone has the highest rate at alkaline pH. The diffusion mechanisms for all the drug release was limited to fickian and non-fickian diffusion mechanisms, without following the carriage (n>0.89) or relaxation transport (n=0.89) mechanisms.

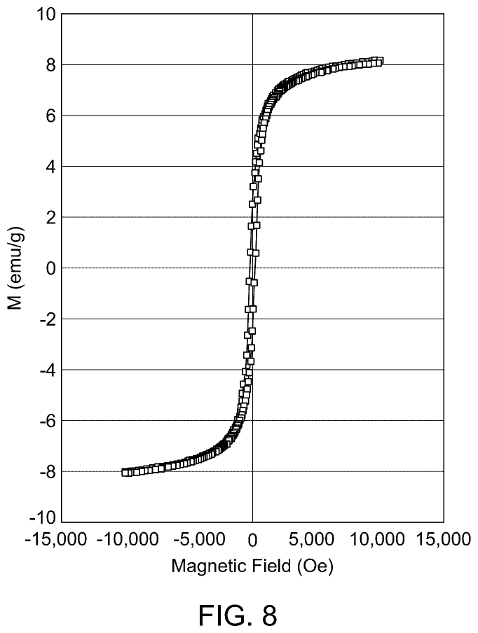

In the nickel ferrite system, Fe³⁺ ions occupied the tetrahedral (A) and octahedral sites (B), while Ni²⁺ occupied the octahedral sites. The anti-parallel moment of ions present in A and B sites leads to the ferrimagnetic property of NiFe₂O₄. As such, the crystalline bulk nickel ferrite achieved a higher saturation magnetization ranging from 40 electromagnetic unit per gram (emu/g) to 60 emu/g. The saturation magnetic properties of nickel ferrite were also influenced by the particle size and non-magnetic surface layers. For instance, nickel ferrite magnetic NPs exhibited superparamagnetism with nanoparticle sizes below 30 nm to 15 nm. Nickel ferrite loaded MCM-41, SBA-15 have been reported to show a lower emu/g. In the present disclosure, the NPs of nickel ferrite formed acquired superparamagnetic property with saturation magnetization of about 8 emu/g as shown in FIG. 8 which evidences the magnetic properties of NiFe₂O₄/Sil nanocomposite. This showed the formation of nanoscale nickel ferrite formation leading to the lower saturation magnetization due to surface effects, such as spin canting, and presence of Sil surface layers.

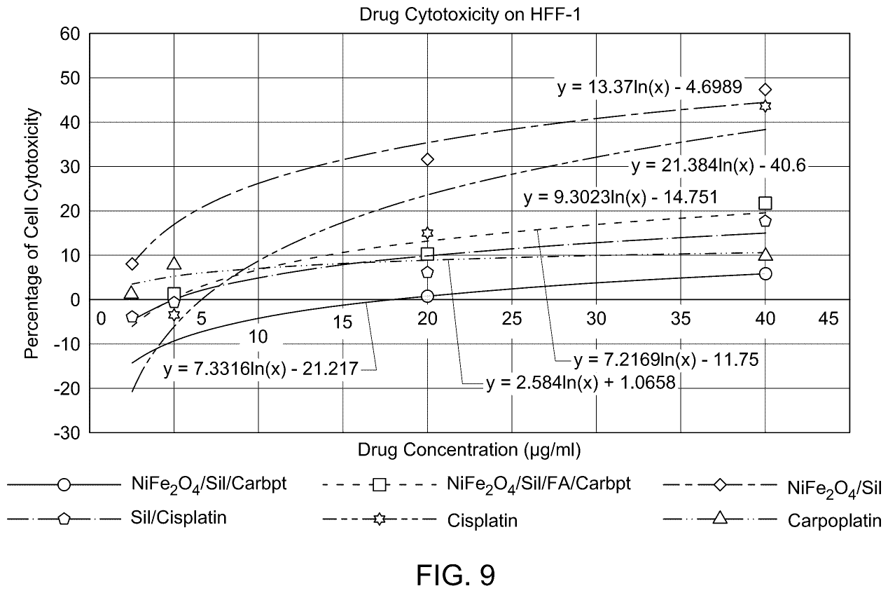

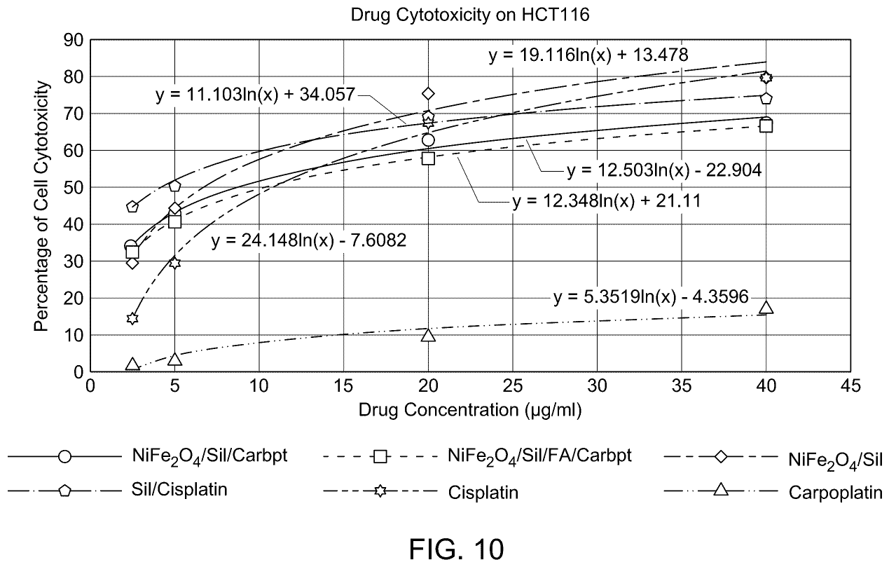

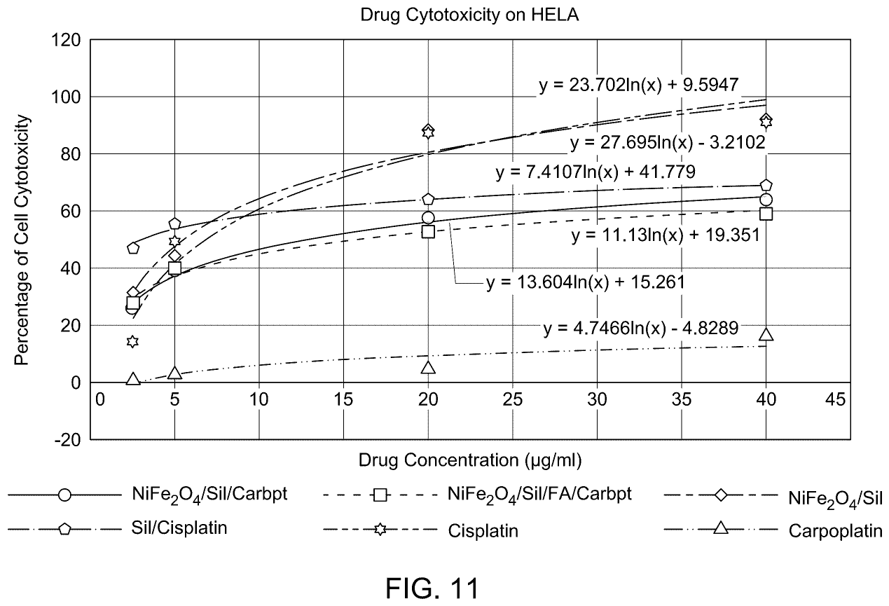

The anticancer activity of controls (Cispt, Carbpt and NiFe₂O₄/Sil) and nanoformulations (NiFe₂O₄/Sil/Carbpt, NiFe₂O₄/Sil/Cispt and NiFe₂O₄/Sil/FA/Carbpt) was assessed using HFF-1, HCT116 cells, and HeLa cells, as shown in FIG. 9, FIG. 10 and FIG. 11. NiFe₂O₄/Sil/Cispt was used for comparative purpose. Both Cispt alone and Cispt bound nanoformulation NiFe₂O₄/Sil/Cispt exhibited significant toxicity against normal cells HFF-1 indicating the non-selective nature of the raw chemo-drug. Remarkably, a significant difference in the LC₅₀ value was observed between Cispt, Carbpt, and their nanoformulations NiFe₂O₄/Sil/Carbpt and NiFe₂O₄/Sil/FA/Carbpt, as listed in Table 3. The cytotoxicity of Carbpt, NiFe₂O₄/Sil/Cispt, NiFe₂O₄/Sil/FA/Carbpt and control nanocarrier alone NiFe₂O₄/Sil exhibited a lower LC₅₀ value towards HFF-1 suggesting a lower toxicity towards normal cells. Conversely, the nanoformulation exhibited a significant toxicity against colon and cervical cells (HeLa and HCT 116). The nanoformulation NiFe₂O₄/Sil/FA/Carbpt showed excellent cytotoxicity comparable to the Cispt based nanoformulation NiFe₂O₄/Sil/Cispt and Cispt alone. The sensitivity of NiFe₂O₄/Sil/Carbpt and NiFe₂O₄/Sil/FA/Carbpt to HeLa and HCT 116 was higher than HFF-1. Further, results suggested the selective toxicity of NiFe₂O₄/Sil/FA/Carbpt towards colon and cervical cancer cells over normal cells. The cell viability of HFF-1 cells remained relatively high even at the higher concentrations of nanoformulations. However, Cispt bound NiFe₂O₄/Sil/Cispt displayed higher sensitivity towards both normal HFF-1 cells and HeLa/HCT 116 cells.

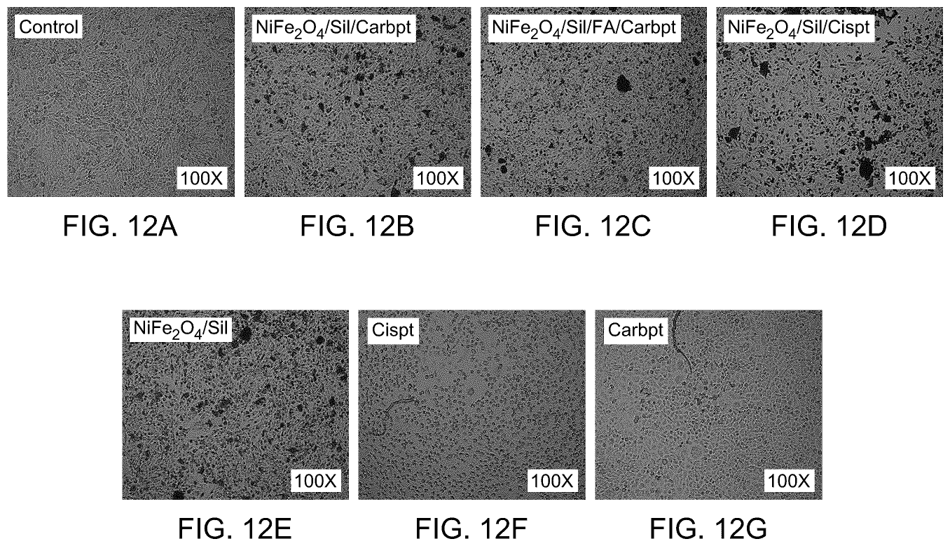

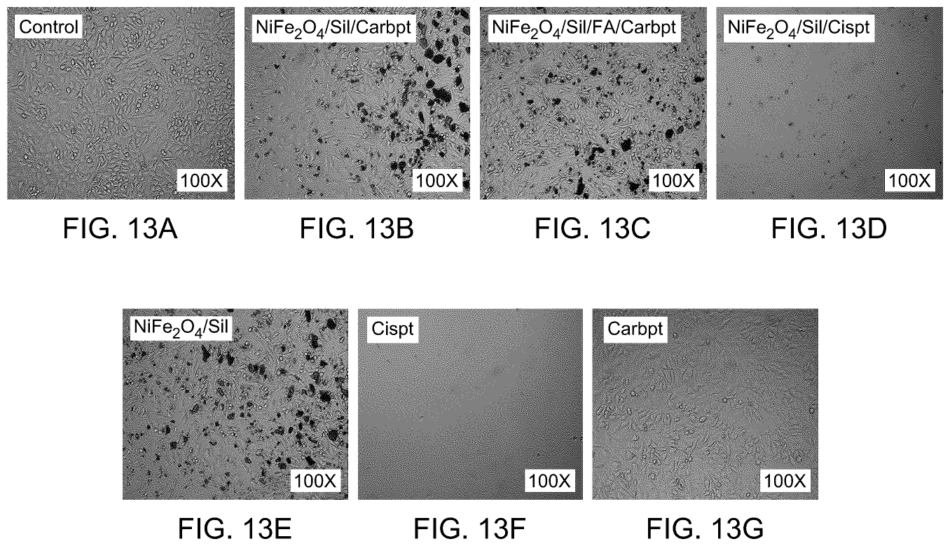

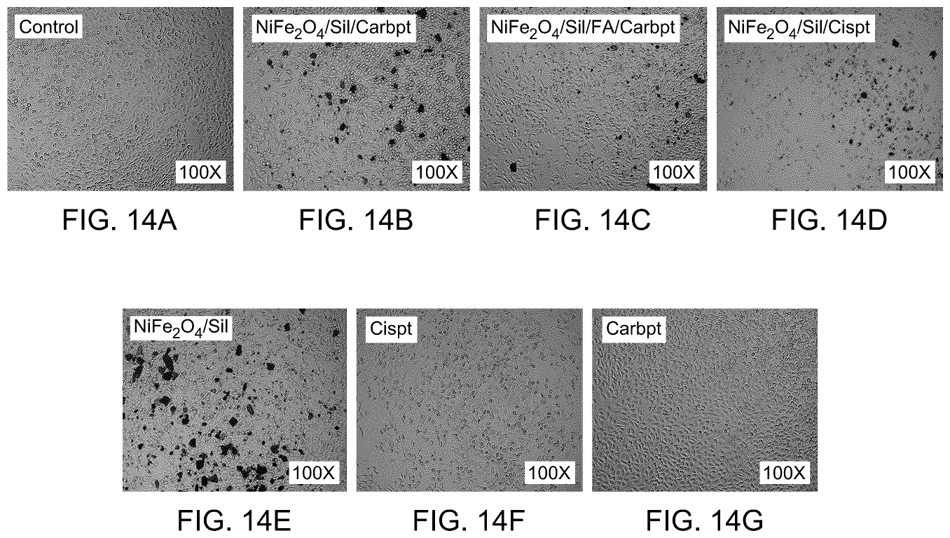

The cell morphology images of HFF-1, HeLa and HCT 116 cells were recorded after 24 hours of treatment with 20 μg/mL and 40 μg/mL. FIG. 12A-12G shows cell morphology images of HFF-1 cells after 24 hours of treatment with 40 μg/mL of control, NiFe₂O₄/Sil/Carbpt, NiFe₂O₄/Sil/FA/Carbpt, NiFe₂O₄/Sil/Cispt, NiFe₂O₄/Sil, Cispt, and Carbpt, respectively. FIG. 13A-13G shows cell morphology images of HELA cells after 24 hours of treatment with 20 μg/mL of control, NiFe₂O₄/Sil/Carbpt, NiFe₂O₄/Sil/FA/Carbpt, NiFe₂O₄/Sil/Cispt, NiFe₂O₄/Sil, Cispt, and Carbpt, respectively. FIG. 14A-14G shows cell morphology images of HCT116 cells after 24 hours of treatment with 20 μg/mL of control, NiFe₂O₄/Sil/Carbpt, NiFe₂O₄/Sil/FA/Carbpt, NiFe₂O₄/Sil/Cispt, NiFe₂O₄/Sil, Cispt, and Carbpt, respectively. The small black dots are the nanoparticles and the brown debris is the dead cells.

The four nanoformulations NiFe₂O₄/Sil/Carbpt, NiFe₂O₄/Sil/FA/Carbpt, NiFe₂O₄/Sil/Cispt, and NiFe₂O₄/Sil showed less toxic effect to normal cells comparable to that of control cells. However, Cispt loaded NiFe₂O₄/Sil (NiFe₂O₄/Sil/Cispt), Cispt and Carbpt confirmed a significant sensitivity towards to normal cells. The nanoformulations showed cell toxicity when treated with both HeLa and colon cell lines. Table 3 hereinbelow provides the LC₅₀ of the evaluated drug delivery systems with respect to HFF-1 cells. The results of Table 3 are presented with an average standard deviation of three independent measurements and the associated p value.

TABLE 3

HFF-1

| Drug Delivery System | LC50 | STD | p value |
|---|---|---|---|
| NiFe₂O₄/SiO₂/Carbpt | 16542.78 | 3060.58 | 0.0145 |
| NiFe₂O₄/SiO₂/FA/Carbpt | 1054.43 | 175.68 | 0.0145 |
| NiFe₂O₄/SiO₂/Cispt | 59.81 | 0.19 | 0.0225 |
| NiFe₂O₄/SiO₂ | 5199.44 | 240.85 | 0.0000 |
| Cispt | 69.19 | 0.34 | 0.0225 |
| Carbpt | 167649139.97 | 5426.17 | 0.5415 |

Table 4 hereinbelow provides the LC₅₀ of the evaluated drug delivery systems with respect to HCT116 cells. The results of Table 4 are presented with an average standard deviation of three independent measurements and the associated p value.

TABLE 4

HCT116

| Drug Delivery System | LC50 | STD | p value |
|---|---|---|---|
| NiFe₂O₄/SiO₂/Carbpt | 8.73 | 0.64 | 0.0013 |
| NiFe₂O₄/SiO₂/FA/Carbpt | 10.38 | 0.56 | 0.0003 |
| NiFe₂O₄/SiO₂/Cispt | 6.76 | 0.30 | 0.0043 |
| NiFe₂O₄/SiO₂ | 4.20 | 0.69 | 0.0043 |
| Cispt | 10.87 | 0.55 | 0.0016 |
| Carbpt | 25772.56 | 3890.81 | 0.0011 |

Table 5 hereinbelow provides the LC₅₀ of the evaluated drug delivery systems with respect to HELA cells. The results of Table 5 are presented with an average standard deviation of three independent measurements and the associated p value.

TABLE 5

| | |
|---|---|
| Drug Delivery | HELA |

| System | LC50 | STD | p value |
|---|---|---|---|
| NiFe₂O₄/SiO₂/Carbpt | 11.69 | 0.73 | 0.0062 |
| NiFe₂O₄/SiO₂/FA/ | 16.37 | 1.55 | 0.0008 |
| Carbpt | | | |
| NiFe₂O₄/SiO₂/Cispt | 5.50 | 0.23 | 0.0092 |
| NiFe₂O₄/SiO₂ | 3.09 | 0.20 | 0.0092 |
| Cispt | 6.83 | 0.20 | 0.0067 |
| Carbpt | 131960.84 | 17783.95 | 0.0008 |

Developing a targeted drug delivery system (DDS) for colon, colorectal and cervical cancer is complicated due to pH variation in the microenvironments. A time-, and pH−-based DDS has however been developed in the present disclosure. Folate is overexpressed in cervical and colon cancer. In the present disclosure, pH−, magnetic-, and FA-based monodispersed spherical silica particles (Sil) were examined for efficacy in delivering carboplatin to targeted cancer cells.

To conclude, the present disclosure develops an effective drug delivery system (DDS) for killing colon cancer cells, colorectal cancer cells and cervical cancer cells. The physico-chemical characteristics of the functionalized nanocomposite confirm the transformation of crystalline to nanosize Carbpt and FA on monodisperse spherical silica particles (Sil). Deposition of NiFe₂O₄ at the external pore surface of Sil was confirmed using BET surface area analysis, which NiFe₂O₄ demonstrates superparamagnetism with a magnetic saturation value of 8 emu/g. A functionalized nanocomposite based on 30 wt. % NiFe₂O₄/spherical silica/Folic acid showed a cervical pH stimuli carboplatin release at pH 5.6, while exhibiting a slow release at a colonic pH of 7.4. The diffusion mechanisms for the carboplatin release from the functionalized nanocomposite was limited to fickian and non-fickian diffusion mechanisms, without following the carriage (n>0.89) or relaxation transport (n=0.89) mechanisms. Nanoformulation cytotoxicity study in in vitro cells showed a lower toxicity to normal cells (HFF-1), while demonstrating toxicitiy to targeted cancer cells (HeLa and HCT 116).

Numerous modifications and variations of the present disclosure are possible in light of the above teachings. It is, therefore, to be understood that within the scope of the appended claims, the invention may be practiced otherwise than as specifically described herein.

1. A method of treating cancer cells to achieve apoptosis, comprising:
contacting the cancer cells with a porous particulate nanocomposite in an amount sufficient to kill the cancer cells;
wherein the porous particulate nanocomposite, comprises:
a magnetic nickel ferrite (NiFe₂O₄); and,
monodisperse spherical silica particles onto which the magnetic nickel ferrite (NiFe₂O₄) is dispersed,
wherein the nanocomposite is functionalized with cis-diammine(cyclobutane-1,1-dicarboxylate-O,O′)platinum(II) (carboplatin) and folic acid, and further wherein the magnetic nickel ferrite (NiFe₂O₄) has an inverse spinel crystal structure;
wherein the cancer cells are cells of a cancer selected from the group consisting of colon cancer, colorectal cancer and cervical cancer.

2. The method of claim 1, wherein the nickel ferrite (NiFe₂O₄) of the porous particulate nanocomposite is superparamagnetic.

3. The method of claim 1, wherein the porous particulate nanocomposite has a surface area of from about 30 to about 50 m²/g, as determined by Brunauer-Emmett-Teller (BET) analysis.

4. The method of claim 1, wherein the porous particulate nanocomposite has a surface area of from about 30 to about 40 m²/g, as determined by Brunauer-Emmett-Teller (BET) analysis.

5. The method of claim 1, wherein the porous particulate nanocomposite has a median pore diameter of from about 10 to about 20 nm, as determined by Barrett-Joyner-Halenda (BJH) desorption analysis.

6. The method of claim 1, wherein the porous particulate nanocomposite has a median pore diameter of from about 15 to about 20 nm, as determined by Barrett-Joyner-Halenda (BJH) desorption analysis.

7. The method of claim 1, wherein the porous particulate nanocomposite has a pore volume of from about 0.1 to about 0.3 cm³/g, as determined by Barrett-Joyner-Halenda (BJH) desorption analysis.

8. The method of claim 1, wherein the porous particulate nanocomposite has a pore volume of from about 0.1 to about 0.2 cm³/g, as determined by Barrett-Joyner-Halenda (BJH) desorption analysis.

9. The method of claim 1, wherein the cis-diammine(cyclobutane-1,1-dicarboxylate-O,O′)platinum(II) is present in an amount of from about 1 to about 10 wt. %, based on the weight of the porous particulate nanocomposite.

10. The method of claim 9, wherein the cis-diammine(cyclobutane-1,1-dicarboxylate-O,O′)platinum(II) is present in an amount of from about 3 to about 7 wt. %, based on the weight of the porous particulate nanocomposite.

11. The method of claim 1, wherein the folic acid is present in an amount of from about 1 to about 10 wt. %, based on the weight of the porous particulate nanocomposite.

12. The method of claim 11, wherein the folic acid is present in an amount of from about 3 to about 7 wt. %, based on the weight of the porous particulate nanocomposite.

13. The method of claim 1, wherein the porous particulate nanocomposite comprises amide bonds, as determined by Fourier Transform Infrared Spectroscopy.

14. The method of claim 1, wherein the Fourier Transform Infrared (FTIR) Spectrum of the porous particulate nanocomposite has an Si—OH peak of lower intensity than that of an FTIR spectrum of a porous particulate nanocomposite of magnetic nickel ferrite (NiFe₂O₄) and monodisperse spherical silica particles which is functionalized only with folic acid.

15. The method of claim 1, wherein according to the Korsmeyer-Peppas equation:
$${M}_{t} / {M}_{∞} = k . {t}^{n}$$
in which: Mₜ/M_{∞} represents the fraction of cis-diammine(cyclobutane-1,1-dicarboxylate-O,O′)platinum(II) released from the porous particulate nanocomposite at time, t
the functionalized nanocomposite has, in a phosphate buffered solution at a pH of about 7.4:
a release constant k of from about 5 to about 30 h⁻ⁿ; and,
a transport exponent n of from about 0.05 to about 0.30.

16. The method of to claim 15, wherein the porous particulate nanocomposite has, in a phosphate buffered solution at a pH of about 7.4:
a release constant k of from about 10 to about 20 h⁻ⁿ; and, a transport exponent n of from about 0.05 to about 0.15.

17. The method of claim 1, further comprising:
calcining a comminuted solid mixture of a nickel (II) salt, an iron (III) salt and silica (SiO₂) at a temperature of from about 700 to about 1000° C. to form a particulate nanocomposite product of magnetic nickel ferrite (NiFe₂O₄) and monodisperse spherical silica particles;
under mixing, adding in a dropwise manner a solution of folic acid in a phosphate buffered saline solution to the particulate nanocomposite product to form a paste;
drying the paste at a temperature of from about 20 to about 50° C. to form a folic acid functionalized particulate nanocomposite intermediate;
under mixing at a temperature of from about-10 to about 20° C., adding a solution of cis-diammine(cyclobutane-1,1-dicarboxylate-O,O′)platinum(II) in normal saline solution to the folic acid functionalized particulate nanocomposite intermediate to form a dispersion of the particulate nanocomposite functionalized with folic acid and cis-diammine(cyclobutane-1,1-dicarboxylate-O,O′)platinum(II); and,
separating the porous particulate nanocomposite from the dispersion.

18. The method of claim 17, wherein:
the nickel (II) salt is selected from the group consisting of nickel sulfate (NiSO₄), nickel nitrate (Ni(NO₃)₂), nickel chloride (NiCl₂) and nickel acetate (Ni(CH₃COO)₂); and,
the iron (III) salt is selected from the group consisting of iron sulfate (Fe₂(SO₄)₃), iron nitrate (Fe(NO₃)₃), iron chloride (FeCl₃) and iron acetate (Fe(CH₃COO)₃).
